(19) United States
(12) Patent Application Publication
(10) Pub. No.: US 2010/0016208 A1
(43) Pub. Date: Jan. 21, 2010

(54) PHOTOACTIVATABLE ANTIMICROBIAL AGENTS

(75) Inventors: Tayyaba Hasan, Arlington, MA (US); Gerald J. Nau, Sewickley, PA (US)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/093,510

(22) PCT Filed: Nov. 15, 2006
(86) PCT No.: PCT/US06/44369

§ 371 Date: Oct. 2, 2008

Related U.S. Application Data
(60) Provisional application No. 60/736,917, filed on Nov. 15, 2005.

(51) Int. Cl. A61K 38/00; C07K 2/00; C07D 501/00; C07D 499/00; C07D 487/00; A61K 31/545; A61K 31/43; A61K 31/407; A61P 31/00

(52) U.S. Cl. 514/2; 530/300; 540/215; 540/304; 540/302; 540/145; 514/200; 514/192; 514/210.09

(57) ABSTRACT

Photoactivatable antimicrobial compounds and methods for the use thereof in the treatment of infections are provided.

### CROSS-REFERENCE TO RELATED APPLICATIONS/PATENTS & INCORPORATION BY REFERENCE

This application claims priority to U.S. Provisional Application Ser. No. 60/736,917, filed Nov. 15, 2005, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Photosensitizers are light-sensitive compounds which undergo a photochemical reaction after the absorption of light quantum. In comparison to healthy cells, malignant and some pre-malignant cells accumulate photosensitizers in a higher concentration, as a result of metabolic processes, and also for a longer period of time. Photosensitizers are activated by laser light of appropriate wavelength and sufficient intensity.

Depending on the type of photosensitizer in question and its charge, accumulation can occur on cell membranes, in mitochrondria, or lysosomes. Damage occurs to membranes through photooxidation of unsaturated fatty acids, lipid-peroxidation and protein-crosslinking (Gomer, C. J., et al. (1989) Radiat. Res. 120:1-18). The inhibition of certain membrane-positioned enzymes (Modica-Napoloitano, J. S., et al. (1990) Cancer Res. 50:7876-7881), a change in the intracellular Ca²⁺-ion concentrations (Hubmer, A., et al. (1996) Photochem. Photobiol. 64:211-215), and the induction of apoptosis (Luo, Y., et al. (1996) Photochem. Photobiol. 63:528-534) can also occur.

Photodynamic therapy (PDT) has been proposed as an attractive method of eliminating oral bacteria and bacteria in topical and gastrointestinal infections because these sites are relatively accessible to targeted illumination (Wilson, M. (1993), J. Appl. Bacteriol. 75:299-306). Photodynamic compounds having improved specificity would be desirable for increasing the safety and efficacy of antibacterial photodynamic therapies acting throughout the body.

### SUMMARY OF THE INVENTION

In one aspect, the invention provides a photosensitizer composition comprising a plurality of photosensitizers that are linked by one or more linkers, wherein said one or more linkers comprise an enzyme cleavage site for an enzyme of a pathogen and wherein said linked photosensitizers are present in an amount sufficient to quench photoactivation of said photosensitizers.

In another aspect, the invention provides a photosensitizer composition comprising a plurality of linked photosensitizers in an amount sufficient to quench photoactivation of the photosensitizers, wherein one or more linkers comprise an enzyme cleavage site for an enzyme of a pathogen, and wherein the composition comprises a targeting moiety. The targeting moiety may direct the composition to a pathogen or a host cell infected with a pathogen. Such an infected host cell may be a macrophage. In one embodiment, the targeting moiety is liposomal. In another embodiment, the targeting moiety comprises a peptide. The peptide may be a small anti-microbial peptide or an active fragment or analog thereof.

In yet another aspect, the invention provides a photosensitizer composition comprising a plurality of photosensitizers and one or more binders effective to quench photoactivation, wherein the photosensitizers are connected to the binder through a linker comprising an enzyme cleavage site for an enzyme of a pathogen. In a further embodiment, the binder is a fluorophore.

In yet another aspect, the invention provides a photosensitizer composition comprising a backbone coupled to a plurality of photosensitizers and one or more binders effective to quench photoactivation, wherein the binders are connected to the backbone through a linker comprising an enzyme cleavage site for an enzyme of a pathogen.

In yet another aspect, the invention provides a photosensitizer composition comprising a backbone coupled to a plurality of photosensitizers and one or more binders effective to quench photoactivation, wherein the photosensitizers are connected to the backbone through a linker comprising an enzyme cleavage site for an enzyme of a pathogen. In one embodiment, the backbone comprises a targeting moiety. In another embodiment, the backbone comprises a polyamino acid. The polyamino acid can be polylysine.

In one embodiment, the enzyme cleavage site of the photosensitizer composition comprises a cephalosporin, a penicillin, a penem, a carbapenem, a monocyclic monobactem, or a fragment thereof. In a further embodiment, the enzyme cleavage site of the photosensitizer composition comprises a cephalosporin, a penicillin, or a fragment thereof. The cephalosporin or penicillin fragment can comprise a beta-lactam ring, and the enzyme cleavage site can be cleaved by a lactamase. In another embodiment, the enzyme cleavage site is a cephalosporin. At least one photosensitizer can be bound at the 3′ position of the cephalosporin.

In yet another embodiment, the pathogen is a Gram (+) bacterial species. The Gram (+) bacterium can be, but are not limited to, Staphylococcus or Enterococcus.

In yet another embodiment, the pathogen is a Gram (−) bacterial species. The Gram (−) bacterium can be, but are not limited to, Escherichia, Haemophilus, Neisseria, Klebsiella, Pasteurella, Proteus, Pseudomonas, Streptophomonas, Burholderia, Acinetobacter, Enterobacter, Serratia, or Salmonella.

Accordingly, the pathogen can be, but is not limited to, Staphylococcus, Enterococcus, Acinetobacter, Enterobacter, Escherichia, Haemophilus, Neisseria, Klebsiella, Pasteurella, Proteus, Pseudomonas, Streptophomonas, Burkholderia, Serratia, or Salmonella spp, Staphylococcus aureus, Staphylococcus epidermis, Enterococcus faecalis, Enterococcus faecium, Escherichia coli, Haemophilus influenzae, Neisseria gonorrhea, Klebsiella pneumoniae, Pasteurella multocida, Proteus mirabilis, Pseudomonas aeruginosa, Stenotrophomonas maltophilia, Burkholderia cepacia, Acinetobacter baumannii, Enterobacter aerogines, Enterobacter cloacae, Serratia marcescens, Salmonella enterica, or Salmonella typhimurium.

In an yet another embodiment, the enzyme cleavage site can comprise D-ala-D-ala (SEQ ID No. 1), and the enzyme cleavage site can be cleaved by a dipeptidase. In a specific embodiment, the pathogen can be Enterococcus faecalis or Enterococcus faecium.

In yet another embodiment, the photosensitizer is a porphyrin. The porphyrin can be, but is not limited to, a porfimer sodium, hematoporphyrin IX, hematoporphyrin ester, dihematoporphyrin ester, synthetic diporphyrin, O-substituted tetraphenyl porphyrin, 3,1-meso tetrakis porphyrin, hydroporphyrin, benzoporphyrin derivative, benzoporphyrin monoacid derivative, monoacid ring derivative, tetracyanoethylene adduct of benzoporphyrin, dimethyl acetylenedicarboxylate adduct of benzoporphyrin, δ-aminolevulinic acid, benzonaphthoporphyrazine, naturally occurring porphyrin, ALA-induced protoporphyrin IX, synthetic dichlorin, bacteriochlorin tetra(hydroxyphenyl)porphyrin, purpurin, octaethylpurpurin derivative, etiopurpurin, tin-etio-purpurin, porphycene, chlorin, chlorin e₆, mono-l-aspartyl derivative of chlorin e₆, di-l-aspartyl derivative of chlorin e₆, tin(IV) chlorin e₆, meta-tetrahydroxyphenylchlorin, chlorin e₆ monoethylendiamine monamide, verdin, zinc methyl pyroverdin, copro II verdin trimethyl ester, deuteroverdin methyl ester, pheophorbide derivative, pyropheophorbide, texaphyrin, lutetium (III) texaphyrin, or gadolinium(III) texaphyrin.

In yet another embodiment, the photosensitizer is a photoactive dye. The photoactive dye can be, but is not limited to, a merocyanine, phthalocyanine, chloroaluminum phthalocyanine, sulfonated aluminum PC, ring-substituted cationic PC, sulfonated AlPc, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, phenothiaziniums such as rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, or thionine.

In yet an another embodiment, the photosensitizer can be, but is not limited to, a Diels-Alder adduct, dimethyl acetylene dicarboxylate adduct, anthracenedione, anthrapyrazole, aminoanthraquinone, phenoxazine dye, chalcogenapyrylium dye, cationic selena, tellurapyrylium derivative, cationic imminium salt, or tetracycline.

In yet another embodiment, the photosensitizer composition comprises a plurality of the same photosensitizer.

In yet another embodiment, a binder is present and connected to the photosensitizer by linkers. In specific embodiments, binder can be a fluorophore or an other photosensitizer.

In yet another aspect, the invention provides a pharmaceutical composition comprising a therapeutically effective amount of a photosensitizer composition of the invention and a pharmaceutically acceptable excipient or carrier.

In yet another aspect, the invention provides a method of decreasing the activity of a pathogen in a subject, said method comprising the steps of: contacting the pathogen with a photosensitizer composition comprising a plurality of photosensitizers that are linked by one or more linkers, wherein said one or more linkers comprise an enzyme cleavage site for an enzyme of a pathogen, and optionally one or more binders, and wherein said linked photosensitizers are present in an amount sufficient to quench photoactivation of said photosensitizers; cleaving one or more linkers to dequench the photosensitizer composition and light-activating the composition to produce a phototoxic species, thereby decreasing the activity of the pathogen in the subject.

In yet another aspect, the invention provides a method for treating an infection in a subject, the method comprising administering to the subject a therapeutically effective amount of a photosensitizer composition of the invention, and light-activating the composition to produce a phototoxic species, thereby treating the infection in the subject

Methods of the invention may further comprise the step of obtaining the photosensitizer composition, linker or binder.

In yet another aspect, the invention provides a packaged pharmaceutical comprising a photosensitizer composition of the invention and associated instructions for using said composition to decrease the activity of the pathogen in a subject in accordance with the methods of the invention.

In yet another aspect, the invention provides a kit for decreasing the activity of a pathogen in a subject in need thereof comprising a photosensitizer composition of the invention and instructions for using the photosensitizer composition to decrease the activity of the pathogen in the subject in accordance with the methods of the invention.

In yet another aspect, the invention provides a method of preparing a linked photosensitizer comprising the steps of: reacting an isocynate derivative of a photosensitizer (e.g., Toluidine Blue O) with a cephalosporin, a penicillin, a carbapenem, a monocyclic mobactem, a polypeptide cleavable by an enzyme of Leishmania, or a derivative or fragment thereof.

In a specific embodiment, the isocynate derivative is an isocynate derivative of Toluidine Blue O. Toluidine Blue O may be prepared by reacting Toluidine Blue O with phosgene in the presence of a base. The base may be diphosgene.

In one embodiment, the isocynate derivative of Toluidine Blue O is reacted with a derivative of a cephalosporin.

In another embodiment, the derivative of cephalosporin is prepared by a method comprising the steps of: i) reacting 7-aminocephalosporanic acid with an amino protecting group; and ii) de-esterifying the amino-protected cephalosporanic acid. The amino protecting group may be

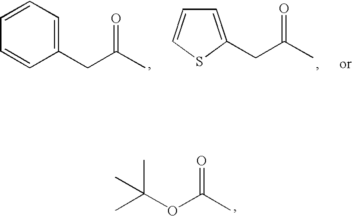

In yet another embodiment, the de-esterified 7-protected cephalosporanic acid is further reacted with an allyl protecting group.

Other aspects of the invention are described in the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 schematically depicts the development of a carbamate-linked photosensitizer (PS) that is inactive (with or without light) while linked and is light-activatable only when released by the β-lactamase enzyme-mediated cleavage.

FIG. 2a shows ¹H NMR spectra obtained for 7-[(2-phenylacetyl)amino]cephalosporanic acid in CDCl₃ as a solvent. FIG. 2b shows ¹H NMR spectrum obtained for 7-[(2-phenylacetyl)amino]3-hydrodxymethy cephalosporanic acid in DMSO-d6 as a solvent. Major proton peaks are marked on the spectra.

FIG. 3 shows MS spectra obtained for (a) 7-[(2-phenylacetyl)amino]3-hydrodxymethy cephalosporanic acid; and (b) cephalosporanic acid-toluidine blue O prodrug.

FIG. 4 shows UV-visible spectra obtained for the photosensitizer (TBO) (black line) vs. the Cephalosporanic acid-photosensitizer prodrug (red line) in ethanol at a concentration of 2.0×10⁻⁵ M.

FIG. 5 shows fluorescence emission spectra obtained for the photosensitizer (TBO) (black line) vs. the Cephalosporanic acid-photosensitizer prodrug (red line) in ethanol at 635 nm excitation.

FIG. 6 shows plots of (a) fluorescence emission vs. wavelength and (b) flurorescence emission vs. time for the Cephalosporanic acid-photosensitizer prodrug, depicting the enzyme-mediated cleavage of the prodrug.

### DETAILED DESCRIPTION OF THE INVENTION

### I. Definitions

The term “photosensitizer” refers to a photoactivatable compound, or a biological precursor thereof, that produces a reactive species (e.g., oxygen) having a photochemical (e.g., cross linking) or phototoxic effect on a cell, cellular component or biomolecule. As used herein, a photosensitizer refers to a substance which, upon irradiation with electromagnetic energy of the appropriate wavelength (e.g., light), produces a cytotoxic effect.

As used herein, the term “fluorescent dye” refers to dyes that are fluorescent when illuminated with light but do not produce reactive species that are phototoxic or otherwise capable of reacting with biomolecules. A photosensitizer will fluoresce when illuminated with a certain wavelength and power of light and also produce reactive species that is phototoxic under the same or different wavelength and power of light. The term “photoactive dye,” as used herein, means that the illuminated photosensitizer produces a fluorescent species, but not necessarily a reactive species in phototoxic amounts (i.e., a phototoxic species). Depending on the wavelength and power of light administered, a photosensitizer can be activated to fluoresce and, therefore, act as a photoactive dye, but not produce a phototoxic species. The wavelength and power of light can be adapted by methods known to those skilled in the art to bring about a phototoxic effect where desired.

As used herein, the term “linker” refers to an agent capable of linking two components of the photosensitizer composition together (e.g., a photosensitizer to another photosensitizer, a photosensitizer to a binder, a photosensitizer to a backbone, a binder to a backbone, a photosensitizer to a targeting moiety, or a binder to a targeting moiety).

As used herein, the term “binder” refers to an agent that absorbs energy from an adjacent, activated photosensitizer or otherwise inactivates the photosensitizer, and, thus, quenches the photosensitizer.

As used herein, the term “enzyme cleavage site” refers to a pathogen-specific amino acid sequence that is cleaved by an enzyme of a pathogen.

As used herein, the terms “peptide”, “polypeptide”, and “protein” are, unless specified otherwise, used interchangeably. Peptides, polypeptides, and proteins used in methods and compositions described herein can be recombinant, purified from natural sources, or chemically synthesized. For example, reference to the use of a bacterial protein or a protein from bacteria, includes the use of recombinantly produced molecules, molecules purified from natural sources, or chemically synthesized molecules.

The term “plurality” refers to at least two, preferably at least about 10 and even more preferably, at least about 20 or more photosensitizer or binder molecules present in a composition of the invention.

The term “subject” is used herein to refer to a living animal, including a human, that carries an unwanted organism, the unwanted organism being the target of the therapeutic methods described herein.

As used herein, “pathogen” or “target organism” means an organism which causes or aggravates a disorder, such as an infection, granuloma, or other adverse immune response.

As used herein, the term “therapeutically effective amount” refers to that amount of a photosensitizer composition that, when administered to a subject, is sufficient to effect a decrease in the activity of a pathogen, as defined herein. Thus, e.g., a therapeutically effective amount of a photosensitizer composition as described herein is a quantity sufficient to result in a decrease in the activity of a pathogen or treatment of an infection mediated by such pathogen, so that the infection is reduced or alleviated.

As used herein, the term “small anti-microbial peptide” (SAMP) refers to a peptide of less than about 60 amino acid residues in length.

As used herein, a “peptide antibiotic” is a linear or cyclic oligopeptide, or an active fragment, or analog thereof, which possesses antibiotic activity against bacterial or fungal species, and which is synthesized enzymatically on a multi-protein complex to which it is attached by a thioether bond. A peptide antibiotic may include non-ribosomal amino acids such as D amino acids, and may include non-amino acid residues such as esters of lactic acid or valeric acid.

The term “obtaining” as in “obtaining” the “photosensitizer composition,” “linker” or “binder,” is intended to include purchasing, synthesizing or otherwise acquiring the elements of the invention.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### II. Compositions of the Invention

A. Photosensitizers

Photosensitizers known in the art are typically selected for use according to: 1) efficacy in delivery, 2) proper localization in target tissues, 3) wavelengths of absorbance, 4) proper excitatory wavelength, 5) purity, and 6) in vivo effects on pharmacokinetics, metabolism, and reduced toxicity.

A photosensitizer for clinical use is optimally amphiphilic, meaning that it shares the opposing properties of being water-soluble, yet hydrophobic. The photosensitizer should be water-soluble in order to pass through the bloodstream systemically, however it should also be hydrophobic enough to pass across cell membranes. Modifications, such as attaching polar residues (amino acids, sugars, and nucleosides) to the hydrophobic porphyrin ring, can alter polarity and partition coefficients to desired levels. Such methods of modification are well known in the art.

In specific embodiments, photosensitizers of the present invention absorb light at a relatively long wavelength, thereby absorbing at low energy. Low-energy light can travel further through tissue than high-energy light, which becomes scattered. Optimal tissue penetration by light occurs between about 650 and about 800 nm. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 nm) or bacteriochlorins found in photosynthetic bacteria (about 750 to about 820 nm).

Photosensitizers of the invention can be any known in the art, and optionally coupled to molecular carriers.

i) Porphyrins and Hydroporphrins

Porphyrins and hydroporphyrins can include, but are not limited to, Photofrin® RTM (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis(o-propionamido phenyl)porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, 5-aminolevulinic acid, benzonaphthoporphyrazines, naturally occurring porphyrins, ALA-induced protoporphyrin IX, synthetic dichlorins, bacteriochlorins of the tetra(hydroxyphenyl)porphyrin series, purpurins, tin and zinc derivatives of octaethylpurpurin, etiopurpurin, tin-etio-purpurin, porphycenes, chlorins, chlorin e₆, mono-l-aspartyl derivative of chlorin e₆, di-l-aspartyl derivative of chlorin e₆, tin(IV) chlorin e₆, meta-tetrahydroxyphenylchlorin, chlorin e₆ monoethylendiamine monamide, verdins such as, but not limited to zinc methyl pyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(III) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generation porphyrins.

Chlorins and bacteriochlorins are also porphyrin derivatives, however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetra hydroxyphenylchlorin (mTHPC) have functional groups to increase solubility. Bacteriochlorins are derived from photosynthetic bacteria and are further red-shifted to about 740 nm. A specific embodiment of the invention uses chlorinₑ₆.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. In a specific embodiment, the photosensitizer can be Antrin®, otherwise known as motexafin lutetium.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin IX is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is MV0633 (Miravant).

In specific embodiments, the photosensitizer has a chemical structure that includes multiple conjugated rings that allow for light absorption and photoactivation. Such specific embodiments include motexafin lutetium (Antrin®) and chlorinₑ₆.

ii) Cyanine and Other Photoactive Dyes

Cyanine and other dyes include but are not limited to a merocyanine, phthalocyanine, chloroaluminum phthalocyanine, sulfonated aluminum PC, ring-substituted cationic PC, sulfonated AlPc, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, phenothiaziniums such as rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, or thionine.

Cyanines are deep blue or purple compounds that are similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

iii) Other Photosensitizers

Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e. mitoxantrone, doxorubicin). Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics such as tetracyclines and fluoroquinolone compounds have demonstrated photoactive properties.

B. Linkers/Enzyme Cleavage Site

Linkers of the invention are capable of linking two components of the photosensitizer composition together (e.g., a photosensitizer to another photosensitizer, a photosensitizer to a binder, a photosensitizer to a backbone, a binder to a backbone, a photosensitizer to a targeting moiety, or a binder to a targeting moiety). Any bond which is capable of linking the components such that they are stable under physiological conditions for the time needed for administration and treatment is suitable, but covalent linkages are preferred. The link between two components may be direct, e.g., where a photosensitizer is linked directly to another photosensitizer, or indirect, e.g., where a photosensitizer is linked to an intermediate, e.g., linked to a backbone, and that intermediate is linked to another photosensitizer. A linker should function under conditions of temperature, pH, salt, solvent system, and other reactants that substantially retain the chemical stability of the photosensitizer, the backbone (if present), and the targeting moiety.

Linkers according to the invention comprise an enzyme cleavage site for a pathogen enzyme. In one aspect of the invention, linker cleavage by a pathogen enzyme causes reduction of the quenching that results from the conformation adopted by the multiple photosensitizers linked to one another. In another aspect, linker cleavage by a pathogen enzyme causes reduction of the quenching that results from inclusion of a binder. Preferably, target cells cause reduction of quenching by the endogenous production of an enzyme which cleaves the linker.

One of the mechanisms utilized by bacteria to become resistant to an antibiotic involves the production of an enzyme that inactivates the antibiotic. An example of this type of resistance constitutes the beta-lactamase enzymes. The beta-lactamase enzymes cleave the four-membered (three carbon, one nitrogen) β-lactam (2-azetidinone) ring that constitutes the unique structural feature shared by the β-lactam antibiotics. This family of antibiotics includes the penicillins, cephalosporins, penems, carbapenems, and monocyclic monobactams. Accordingly, in specific embodiments, the linker comprises a penicillin, a cephalosporin, a carbapenem, or a monocyclic monobactam or a fragment thereof (e.g. comprising a beta-lactam ring).

Another example of this type of resistance constitutes VanX, a dipeptidase that cleaves D-Ala-D-Ala and catalyzes hydrolysis of the D-alanyl-D-alanine dipeptide normally used in wild-type peptidoglycan biosynthesis. VanX-related proteins are involved in the production of a variant peptidoglycan (D-alanine-D-lactate) that results in resistance of pathogenic bacteria to the antibiotic vancomycin. D-ala-D-ala peptidase has been found to be contained in the operon that encodes vancomycin resistance in Enterococcus faecalis and Enterococcus faecium.

A linker can link components without the addition to the linked components of elements of the linker. Other linkers result in the addition of elements of the linker to the linked components. For example, linkers can be cross-linking agents that are homo- or hetero-bifunctional, and wherein one or more atomic components of the agent can be retained in the composition.

Many linkers react with an amine and a carboxylate, to form an amide, or an alcohol and a carboxylate to form an ester. Linkers are known in the art, see, e.g., M. Bodansky, “Principles of Peptide Synthesis”, 2nd ed., referenced herein, and T. Greene and P. Wuts, “Protective Groups in Organic Synthesis,” 2nd Ed, 1991, John Wiley, NY. Linkers should link component moieties stably, but such that there is only minimal or no denaturation or deactivation of the photosensitizer or other linked component.

The photosensitizer compositions of the invention can be prepared by linking the photosensitizers to one another or to other components using methods known in the art. A variety of linkers, including cross-linking agents, can be used for covalent conjugation. Examples of cross-linking agents include N,N′-dicyclohexylcarbodiimide (DCC), N-succinimidyl-S-acetyl-thioacetate (SATA), N-succinimidyl-3-(2-pyridyldithio)propionate (SPDP), ortho-phenylenedinaleimide (o-PDM), and sulfosuccinimidyl 4-(N-maleimidomethyl)cyclohexane-l-carboxylate (sulfo-SMCC) (Karpovsky et al. (1984) J. Exp. Med. 160:1686; Liu, M A et al. (1985) Proc. Natl. Acad. Sci. USA 82:8648). Other methods include those described by Paulus and Behring (1985) Ins. Mitt., 78:118-132; Brennan et al. (1985) Science 229:81-83 and Glennie et al., (1987) J. Immunol, 139:2367-2375. A large number of linkers for peptides and proteins, along with buffers, solvents, and methods of use, are described in the Pierce Chemical Co. catalog, pages T-155 to T-200, 1994 (3747 N. Meridian Rd., Rockford Ill., 61105, U.S.A.; Pierce Europe B.V., P.O. Box 1512, 3260 BA Oud Beijerland, The Netherlands), the contents of which are hereby incorporated by reference.

DCC is a useful linker (Pierce #20320; Rockland, Ill.). DCC (N,N′-dicyclohexylcarbodiimide) is a carboxy-reactive cross-linker commonly used as a linker in peptide synthesis. Another useful cross-linking agent is SPDP (Pierce #21557), a heterobifunctional cross-linker for use with primary amines and sulfhydryl groups. SPDP produces cleavable cross-linking such that, upon further reaction, the agent is eliminated, so the photosensitizer can be linked directly to a backbone or molecular carrier. Other useful linking agents are SATA (Pierce #26102) for introduction of blocked SH groups for two-step cross-linking (Pierce #26103), and sulfo-SMCC (Pierce #22322), reactive towards amines and sulfhydryls. Other cross-linking and coupling agents are also available from Pierce Chemical Co. (Rockford, Ill.).

Additional useful linking agents are hydrazines or hydrazine derivatives, compounds that are very soluble in water and soluble in alcohol. Hydrazines are corrosive and strong reducing agents, though they constitute weaker bases than ammonia. Hydrazines are dibasic and form many salts, e.g., mono- and di-hydrochlorides, mono- and di-nitrates, and two sulfates. The hydrazine resin has been found to be a novel and highly useful platform for polyamide synthesis. The hydrazine resin is stable to elevated coupling temperatures, yet is cleaved rapidly at moderate temperatures by a wide range of nucleophiles following a mild and selective oxidation protocol.

Additional compounds and processes, particularly those involving a Schiff base as an intermediate, for conjugation of proteins to other proteins or to other compositions, for example, to reporter groups or to chelators for metal ion labeling of a protein, are disclosed in EP 243,929 A2 (published Nov. 4, 1987).

Photosensitizers which contain carboxyl groups can be joined to lysine s-amino groups in target polypeptides either by preformed reactive esters (such as N-hydroxy succinimide ester) or esters conjugated in situ by a carbodiimide-mediated reaction. The same applies to photosensitizers that contain sulfonic acid groups, which can be transformed to sulfonyl chlorides, which react with amino groups. Photosensitizers that have carboxyl groups can be joined to amino groups on the polypeptide by an in situ carbodiimide method or by hydrazine or hydrazine derivatives. Photosensitizers can also be attached to hydroxyl groups, of serine or threonine residues or to sulfhydryl groups, of serine or threonine residues or to sulfhydryl groups of cysteine residues.

Methods of joining components of a composition can use heterobifunctional cross linking reagents. These agents bind a functional group in one chain and a different functional group in a second chain. These functional groups typically are amino, carboxyl, sulfhydryl, and aldehyde. There are many permutations of appropriate moieties that will react with these groups and with differently formulated structures, to join them together (described in the Pierce Catalog and Merrifield et al. (1994) Ciba Found Symp. 186:5-20).

Generally, the photosensitizer compositions of the invention can be prepared by linking the photosensitizer to another photosensitizer, a binder, a targeting moiety, and/or a backbone using methods described in the following Examples or by methods known in the art. A variety of linkers can be used for covalent conjugation.

Yield from linking reactions can be assessed by spectroscopy of product eluting from a chromatographic fractionation in the final step of purification. The presence of unlinked photosensitizer and reaction products containing the photosensitizer can be followed by the physical property that the photosensitizer absorbs light at a characteristic wavelength and extinction coefficient, so incorporation into products can be monitored by absorbance at that wavelength or a similar wavelength. Linking of one or more photosensitizer molecules to another or to a binder or to a targeting moiety or to a backbone shifts the peak of absorbance in the elution profile in fractions eluted using sizing gel chromatography, e.g., with the appropriate choice of Sephadex G50, G100, or G200 or other such matrices (Pharmacia-Biotech, Piscataway N.J.). Choice of appropriate sizing gel, for example Sephadex gel, can be determined by that gel in which the photosensitizer elutes in a fraction beyond the excluded volume of material too large to interact with the bead, i.e., the uncoupled starting photosensitizer composition interacts to some extent with the fractionation bead and is concomitantly retarded to some extent.

The correct useful gel can be predicted from the molecular weight of the uncoupled photosensitizer. The successful reaction products of photosensitizer compositions coupled to additional moieties generally have characteristic higher molecular weights, causing them to interact with the chromatographic bead to a lesser extent, and thus appear in fractions eluting earlier than fractions containing the uncoupled photosensitizer substrate. Unreacted photosensitizer substrate generally appears in fractions characteristic of the starting material, and the yield from each reaction can thus be assessed both from size of the peak of larger molecular weight material, and the decrease in the peak of characteristic starting material. The area under the peak of the product fractions is converted to the size of the yield using the molar extinction coefficient.

The product can be analyzed using NMR, integrating areas of appropriate product peaks, to determine relative yields with different linkers. A red shift in absorption of a photosensitizer of several nm has often been observed following coupling to a polyamino acid. Linking to a larger moiety such as a protein might produces a comparable shift, as linking to an antibody resulted in a shift of about 3-5 nm in that direction compared to absorption of the free photosensitizer. Relevant absorption maxima and extinction coefficients in 0.1M NaOH/1% SDS are, for chlorin e6, 400 nm and 150,000 M⁻¹, cm⁻¹, and for benzoporphyrin derivative, 430 nm and 61,000 M⁻¹, cm⁻¹.

C. Binders

The binder may, without limitation, be a peptide, a cyclic peptide, a polypeptide, a peptidomimetic, a protein, a fusion protein, a hybrid molecule or a dimer, multimer, or a conjugate of the above that binds or quenches, and, thus, may inhibit, suppress, neutralize, or decrease activity of, the photosensitizer. The binder may include, without limitation, a naturally occurring inhibitor, a receptor, a soluble receptor, an antibody, a polyclonal antibody, a monoclonal antibody, a bispecific antibody, an antibody fragment, a single chain antibody, anti-idiotype antibodies, a peptabody, a peptide, an oligopeptides, an oligonucleotide, a cyclic peptide (i.e., a peptide that is circular in nature), a peptide-lipid conjugate, a hormone, an antigen, an epitope, a receptor, a chemokine, a nucleic acid, a ligand or a dimer, multimer, or a conjugate of the above. Naturally occurring binders are binders that quench the photosensitizer and are found in nature.

In one aspect, the binder is a fluorophore. The property that renders a fluorophore (or any other binder) a suitable quencher is the capability of absorbing energy from the activated photosensitizer.

Fluorophores of the present invention can be any known in the art, including photosensitizers, fluorescent dyes, and photoactive dyes.

Photosensitizers can be any known in the art, as previously described. For example, hematoporphyrin derivatives have been used as fluorescent probes to investigate the development of human atherosclerotic plaques (Spokojny (1986) J. Am. Coll. Cardiol. 8:1387-1392). Ideally, the photosensitizer acting as a binder has a different excitation wavelength than the photosensitizer acting to produce a cytotoxic effect on the pathogen or host cell infected with the pathogen.

Fluorescent dyes of the present invention can be any known in the art, including, but not limited to 6-carboxy-4′,5′-dichloro-2′,7′-dimethoxyfluorescein succinimidyl ester; 5-(and-6)-carboxyeosin; 5-carboxyfluorescein; 6-carboxyfluorescein; 5-(and-6)-carboxyfluorescein; 5-carboxyfluorescein-bis-(5-carboxymethoxy-2-nitrobenzyl)ether, -alanine-carboxamide, or succinimidyl ester; 5-carboxyfluorescein succinimidyl ester; 6-carboxyfluorescein succinimidyl ester; 5-(and-6)-carboxyfluorescein succinimidyl ester; 5-(4,6-dichlorotriazinyl)aminofluorescein; 2′,7′-difluorofluorescein; eosin-5-isothiocyanate; erythrosin-5-isothiocyanate; 6-(fluorescein-5-carboxamido)hexanoic acid or succinimidyl ester; 6-(fluorescein-5-(and-6)-carboxamido)hexanoic acid or succinimidyl ester; fluorescein-5-EX succinimidyl ester; fluorescein-5-isothiocyanate; fluorescein-6-isothiocyanate; Oregon Green® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidyl ester or triethylammonium salt; Oregon Green® 514 carboxylic acid; Oregon Green® 514 carboxylic acid or succinimidyl ester; Rhodamine Green™ carboxylic acid, succinimidyl ester or hydrochloride; Rhodamine Green™ carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl)sulfonerhodamine or di(succinimidyl ester); 5-(and-6)-carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; NANOGOLD® mono(sulfosuccinimidyl ester); QSY® 21 carboxylic acid or succinimidyl ester; QSY® 7 carboxylic acid or succinimidyl ester; Rhodamine Red™-X succinimidyl ester; 6-(tetramethylrhodamine-5- (and-6)-carboxamido)hexanoic acid succinimidyl ester; tetramethylrhodamine-5-isothiocyanate; tetramethylrhodamine-6-isothiocyanate; tetramethylrhodamine-5-(and-6)-isothiocyanate; Texas Red® sulfonyl; Texas Red® sulfonyl chloride; Texas Red®-X STP ester or sodium salt; Texas Red®-X succinimidyl ester; Texas Red®-X succinimidyl ester; and X-rhodamine-5-(and-6)-isothiocyanate.

Fluorescent dyes of the present invention can also be, for example, bodipy dyes commercially available from Molecular Probes, including, but not limited to BODIPY® FL; BODIPY® TMR STP ester; BODIPY® TR-X STP ester; BODIPY® 630/650-X STP ester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoic acid; 4,4-difluoro-5,7-dimethyl-4-bora-3a,4a-diaza-s-indacene-3-pentanoic acid succinimidyl ester; 4,4-difluoro-5,7-dimethyl-4-bora-3a,4a-diaza-s-indacene-3-propionic acid; 4,4-difluoro-5,7-dimethyl-4-bora-3a,4a-diaza-s-indacene-3-propionic acid succinimidyl ester; 4,4-difluoro-5,7-dimethyl-4-bora-3a,4a-diaza-s-indacene-3-propionic acid sulfosuccinimidyl ester or sodium salt; 6-((4,4-difluoro-5,7-dimethyl-4-bora-3a,4a-diaza-s-indacene-3-propionyl)amino)hexanoic acid; 6-((4,4-difluoro-5,7-dimethyl-4-bora-3a,4a-diaza-s-indacene-3-propionyl)amino)hexanoic acid or succinimidyl ester; N-(4,4-difluoro-5,7-dimethyl-4-bora-3a,4a-diaza-s-indacene-3-propionyl)cysteic acid, succinimidyl ester or triethylammonium salt; 6-4,4-difluoro-1,3-dimethyl-5-(4-methoxyphenyl)-4-bora-3a,4a4,4-difluoro-5,7-diphenyl-4-bora-3a,4a-diaza-s-indacene-3-propionic acid; 4,4-difluoro-5,7-diphenyl-4-bora-3a,4a-diaza-s-indacene-3-propionic acid succinimidyl ester; 4,4-difluoro-5-phenyl-4-bora-3a,4a-diaza-s-indacene-3-propionic acid succinimidyl ester; 6-((4,4-difluoro-5-phenyl-4-bora-3a,4a-diaza-s-indacene-3-propionyl)amino)hexanoic acid or succinimidyl ester; 4,4-difluoro-5-(4-phenyl-1,3-butadienyl)-4-bora-3a,4a-diaza-s-indacene-3-propionic acid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoic acid or succinimidyl ester; 4,4-difluoro-5-styryl-4-bora-3a,4a-diaza-s-indacene-3-propionic acid; 4,4-difluoro-5-styryl-4-bora-3a,4a-diaza-s-indacene-3-propionic acid succinimidyl ester; 4,4-difluoro-1,3,5,7-tetramethyl-4-bora-3a,4a-diaza-s-indacene-8-propionic acid; 4,4-difluoro-1,3,5,7-tetramethyl-4-bora-3a,4a-diaza-s-indacene-8-propionic acid succinimidyl ester; 4,4-difluoro-5-(2-thienyl)-4-bora-3a,4a-diaza-s-indacene-3-propionic acid succinimidyl ester; 6-(((4-(4,4-difluoro-5-(2-thienyl)-4-bora-3a,4a-diaza s-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester.

Fluorescent dyes the present invention can also be, for example, alexa fluor dyes commercially available from Molecular Probes, including but not limited to Alexa Fluor® 350 carboxylic acid; Alexa Fluor® 430 carboxylic acid; Alexa Fluor® 488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor® 594 carboxylic acid; Alexa Fluor® 633 carboxylic acid; Alexa Fluor® 647 carboxylic acid; Alexa Fluor® 660 carboxylic acid; and Alexa Fluor® 680 carboxylic acid.

Fluorescent dyes the present invention can also be, for example, cy dyes commercially available from Amersham-Pharmacia Biotech, including, but not limited to Cy3 NHS ester; Cy 5 NHS ester; Cy5.5 NHS ester; and Cy 7 NHS ester.

Photoactive dyes of the present invention can be any photosensitizer known in the art which will fluoresce but not necessarily produce a reactive species in phototoxic amounts when illuminated. Depending on the wavelength and power of light administered, a photosensitizer can be activated to fluoresce and, therefore, act as a photoactive dye, but not produce a phototoxic effect unless, in some cases, the wavelength and power of light is suitably adapted to induce a phototoxic effect.

Throughout this specification, any reference to a binder should be construed to refer to each of the binders identified and contemplated herein and to each biologically equivalent molecule. “Biologically equivalent” means compositions of the present invention which are capable of preventing action of the photosensitizer in a similar fashion, but not necessarily to the same degree.

D. Targeting Moieties

To increase the specificity of the photosensitizer composition for its target a targeting moiety may be included therein. Targeting moieties include antibody and antibody fragments, peptides, and hormones. In one aspect of the invention, the targeting moiety can be a polypeptide (e.g., a human polypeptide such as poly-lysine or serum albumin). Alternatively, the targeting moiety can be a small anti-microbial peptide (i.e. a peptide containing less than 60 amino acid residues). Histatins, defensins, cecropins, magainins, Gram positive bacteriocins, and peptide antibiotics which meet this limitation are SAMP's. Many SAMP's are in the range of 20-40 amino acid residues in length. SAMP's are naturally occurring peptides, and are made by a wide variety of organisms. SAMP's are NPM's. Many SAMP's have a broad spectrum of antimicrobial activity, and, e.g., can kill more than one species, and in some cases can kill distantly related species, e.g. Gram negative and Gram positive bacterial species.

The targeting moiety binds to a defined population of cells. It may bind a receptor, an oligonucleotide, an enzymatic substrate, an antigenic determinant, or other binding site present on or in the target cell population. Accordingly, the targeting moiety can be a molecule or a macromolecular structure that targets specific cells, for example, macrophages, or that interacts with a pathogen. Some photosensitizers target macrophages directly (see, e.g., Korbelik et al., Cancer Res. 51:2251-2255, 1991).

Moieties, a target moiety alone, or incorporated into a conjugate, as in a supramolecular structure (e.g., a liposome, a micelle, a lipid vesicle, or the like), can be used to specifically target macrophages by certain receptors. Thus, a ligand for such receptors can be used as a targeting moiety. For example, the following receptors can be used to target macrophages: the complement receptor (Rieu et al., J. Cell Biol. 127:2081-2091, 1994), the scavenger receptor (Brasseur et al., Photochem. Photobiol. 69:345-352, 1999; Suzuki et al., Nature 386:292-296, 1997; Sarkar et al., Mol. Cell. Biochem. 156:109-116, 1996), the transferrin receptor (Dreier et al., Bioconjug. Chem. 9:482-489, 1998; Hamblin et al., J. Photochem. Photobiol. 26:4556, 1994; Clemens et al., J. Exp. Med. 184:1349-1355, 1996), the Fc receptor (Rojanasakul et al., Pharm. Res. 11:1731-1733, 1994; Harrison et al., Pharm Res. 11:1110-4, 1994). The mannose receptor is particularly important for macrophage recognition of foreign material and has been used successfully to target molecules to macrophages (Frankel et al., Carbohydr. Res. 300:251-258, 1997; Chakrabarty et al., J. Protozool. 37:358-364, 1990; Mistry et al., Lancet 348:1555-1559, 1996; Liang et al., Biochim. Biopys. Acta 1279:227-234, 1996; Sarkar et al., Mol. Cell Biochem. 156:109-116, 1996). Toll or toll-like receptors are also present on macrophages and are useful targets (Brightbill et al., Science 285:732-736, 1999).

Moieties that can be in the photosensitizer compositions of the invention in order to target the latter to macrophages include low density lipoproteins (Mankertz et al., Biochem. Biophys. Res. Commun. 240:112-115, 1997; von Baeyer et al., Int. J. Clin. Pharmacol. Ther. Toxicol. 31:382-386, 1993), very low density lipoproteins (Tabas et al., J. Cell Biol. 115:1547-1560, 1991), mannose residues (as mentioned above) and other carbohydrate moieties (Pittet et al., Nucl. Med. Biol. 22:355-365, 1995), poly-cationic molecules (e.g., poly-Llysine; Hamblin et al., J. Photochem. Photobiol. 26:45-56, 1994), emulsions (Khopade et al., Pharmazie 51:558-562, 1996), aggregated albumin (Hamblin et al., J. Photochem. Photobiol. 26:45-56, 1994), biodegradable microspheres (Oettinger et al., J. Interferon Cytokine Res. 19:33-40, 1999), non-biodegradable microspheres (Schroder, Methods Enzymol 112:116-128, 1985), nanoparticles (Lobenberg et al., AIDS Res. Hum. Retroviruses 12:1709-1715, 1996); Venier-Julienne et al., J. Drug Target. 3:23-29, 1995; Schafer et al., J. Microencapsul. 11:261-269, 1994; Gaspar et al., Ann. Trop. Med. Parasitol 86:41-49, 1992), liposomes (Bakker-Woudenberg et al. J. Drug Target. 2:363-371, 1994; Meyers et al., Exp. Lung Res. 19:1-19, 1993; Betageri et al., J. Pharm. Pharmacol. 45:48-53, 1993; Muller et al., Biochim. Biophys. Acta. 986:97-105, 1989; Kole et al., J. Infect. Dis. 180:811-820, 1999), macrophage-specific cytokines (Biragyn et al., Nat. Biotechnol. 17:253-258, 1999; Chan et al., Blood 86:2732-2740, 1995), erythrocytes (Magnani et al., J. Leukoc. Biol. 185:717-730, 1997), antibodies recognizing components of the tuberculous phagosome like Nrampl (Gruenheid et al., J. Exp. Med. 185:717-730, 1997), a 2-macroglobulin (Chu et al., J. Immunol. 152:1538-1545, 1994).

A targeting moiety can be directed to the infectious pathogen. In addition, certain structural features of enzymes can be targeted, such as the hydrophobic pocket of the Mycobacterium tuberculosis enzyme inhA (Dessen, et al.(1995) Science 267:1638-1641). Alternatively, host molecules that target the bacteria, such as anti-microbial peptides (e.g., granulysin), can be used in photosensitizer compositions of the invention (Stenger et al., Science 282:121-125, 1998).

A targeting moiety can be used alone or in combination, particularly to target both macrophages and the intracellular pathogen. Manipulations of the host cell can also complement the photosensitizer (Collins et al., J. Cell Sci. 110:191-200, 1997; Korbelik et al., Br. J. Cancer 75:202-207, 1997; Krosl et al., Cancer Res. 56:3281-3286, 1996).

The targeting moiety can be a polypeptide. The polypeptide may be linear, branched, or cyclic. The targeting moiety can include a polypeptide having an affinity for a polysaccharide target, for example, a lectin (such as a seed, bean, root, bark, seaweed, fungal, bacterial, or invertebrate lectin). Particularly useful lectins include concanavalin A, which is obtained from jack beans, and lectins obtained from the lentil, Lens culinaris.

Desirable characteristics for the targeting moieties include: specificity for one or more unwanted target organisms or components thereof (e.g. cell surface receptors), affinity and avidity for such organisms, and stability with respect to conditions of coupling reactions and the physiology of the organ or tissue of use. Specificity need not be narrowly defined, e.g., it may be desirable for a targeting molecule to have affinity for a broad range of target organisms, such as all Gram negative bacteria. The targeting moiety, when incorporated into a composition of the invention, should be nontoxic to the cells of the subject.

Targeting moieties can be selected from the sequences of naturally occurring proteins and peptides, from variants of these peptides, and from biologically or chemically synthesized peptides. Naturally occurring peptides which have affinity for one or more target organism can provide sequences from which additional peptides with desired properties, e.g., increased affinity or specificity, can be synthesized individually or as members of a library of related peptides. Such peptides can be selected on the basis of affinity for the target organism.

Naturally occurring peptides with affinity for target organisms useful in methods and compounds of the invention, include aptomers, salivary proteins, e.g., histatins, microbially-elaborated proteins, e.g., bacteriocins, peptides that bind and/or kill species that are closely related to the producing strains; and proteins produced by animal species such as defensins, which are produced by mammals, and the cecropins and magainins, produced by moths and amphibia, respectively.

As mentioned briefly above, histatins, defensins, cecropins and magainins are examples of a class of polypeptides found widely in nature, which share the characteristics of small size (generally approximately 30 amino acid residues, and between 10 residues and 50 residues), broad specificity of anti-microbial activity, and low affinity for target organisms.

Histatins are a family of histidine-rich cationic polypeptides which have bactericidal and candidacidal properties and are constituents of normal human saliva (Oppenheim, G. G. et al., J. Biol. chem. 263:7472-747, 1988). Their mechanism of action is thought to involve a combination of alpha-helical conformation and cationic charge leading them to insert between the polar head groups in the bacterial cell wall (Raj, P. A. et al., J. Biol. Chem. 269:9610-9619, 1994).

Bacteriocins, which are proteins produced by bacteria and which kill other strains and species of bacteria (Jack, R. W. et al., Microbiol. Rev. 59:171-200, 1995) can be used as targeting moieties. An exemplary Gram positive bacteriocin is nisin, produced by Lactococcus lactis and accorded GRAS status (generally regarded as safe) by the Food and Drug Administration for application to food preservation.

The bacteriocins nisin, subtilin, epidermin, gallidermin, salivarin, and lacticin exemplify the “lantibiotic” class of Gram positive bacteriocin, which is defined as a bacteriocin in which one or more cysteine residues are linked to a dehydrated serine or threonine to form a thioether-linked residue known as lanthionine (Lan) or threo-.beta.-methyllanthionine (MeLan). These are post-translational modifications found in these anti-microbial peptides by the producing cell. Lantibiotics contain leader peptide sequences of 18-24 residues, which are cleaved to yield an active antimicrobial peptide of about 22-35 residues. Growth of the producing bacterial species, and preparation and purification of bacteriocins are performed by published procedures and techniques which can be carried out by one of skill in the art. For example, Yang, R. et al., Appl. and Env. Microbiol 58: 3355-3359, 1992, describe purification of bacteriocins from each of 4 genera of lactic acid bacteria, by optimizing absorption onto the producing cells, followed by use of low pH for selective elution of greatly enriched bacteriocin fractions. Mutant forms of each of the bacteriocins nisin, produced by Lactococcus lactis, and of subtilin, produced by Bacillus subtilis have more desirable properties than the parental wild-type forms (Liu, W. and N. Hansen, J. Biol. Chem. 267:25,078-25,085, 1992). Procedures for isolation of appropriate genes and for mutagenesis and selection of strains carrying desirable mutations are found in Maniatis, T. et al, 1982, Molecular Cloning: a Laboratory Manual, Cold Spring Harbor Press, Cold Spring Harbor, N.Y., and in the subsequent second edition, Sambrook, J. et al., 1989.

Anti-microbial peptides are produced by a variety of animals (Saberwal, G. and R. Nagaraj, Biochim. Biophys. Act. 1197:109-131, 1994). An example is a peptide of the cecropin family produced by Cecropia moths. Several cecropins contain 37 residues, of which 6 are lysine. Cecropins are active against both Gram positive and Grain negative bacteria. Other insect-produced peptides include apidaecin (from honeybees), andropin (from fruit flies), and cecropin family members from bumble bees, fruit flies, and other insects.

The defensins are produced by mammals, including humans, and are generally about 29-34 residues in length, and the magainins (about 23 residues) are produced by amphibia such as Xenopus laevis. Defensins from human (HNP-1,-2,-3 and 4), guinea pig (GPNP), rabbit (NP-1, -2, -3A, -3B, -4 and -5) and rat (NP-1, -2, -3 and -4) share a significant number of regions of homology. Defensins can have antimicrobial activity against Gram positive bacteria or Gram negative bacteria and fungi, with minimal inhibitory concentrations in the mM range. Rabbit NP-1 and NP-2 are more potent antibacterial agents than others in this family. Other mammalian anti-microbial peptides include murine cryptdin, bovine granulocyte bactenecin and indolicidin, and seminal-plasmin from bovine semen. Additional amphibial anti-microbials include PGLA, XPF, LPF, CPG, PGQ, bombinin from Bombina variegata, the bombinin-like peptides BLP-1, -2, -3 and -4 from B. orientalis, and brevinins from Rana esculenta. Invertebrates such as the horseshoe crab can be a source of anti-microbial peptides such as the tachyplesins (I, II and III) and the polyphemusins (I and II).

Peptides in these families of antimicrobial agents are generally cationic, and can have a broad antimicrobial spectrum, including both antibacterial and antifungal activities. The addition of positively charged residues can enhance antimicrobial specific activity several fold. The positive charges are thought to assist in the insertion of the peptides into the membranes of the susceptible organisms, in which context the peptide molecules can form pores and cause efflux of ions and other metabolites. Structural studies of the Moses sole fish neurotoxin 33 residue peptide pardaxin, for example, reveals that succinylated pardaxin inserts into erythrocyte and model membranes more slowly than unmodified pardaxin. (Shai, Y et al., J. Biol. Chem. 265: 20,202-20, 209, 1990). The positively charged magainin molecule can disrupt both the metabolism of E. coli and the electric potential of the mitochondrion (Westerhoff, H. V., et al., Proc. Natl. Acad. Sci. 86:6597-6601, 1989).

Novel peptides, for example, a cecropin-melittin hybrid, and synthetic Denantiomers have antimicrobial activity (Merrifield, R. B. et al., “Antimicrobial peptides,” Ciba Foundation Symp. 186, John Wiley, Chichester, pp. 5-26, 1994). One such synthetic cecropin-melittin peptide is 5-fold more active against Mycobacterium smegmatis than rifampin.

Targeting moieties can be plant proteins with affinities for particular target organisms, for example, a member of the lectin protein family with affinity for polysaccharides. Targeting moieties can be synthetic peptides, such as polylysine, polyarginine, polyornithine, and synthetic heteropolypeptides that comprise substantial proportions of such positively charged amino acid residues. Such peptides can be chemically synthesized or produced biologically in recombinant organisms, in which case the targeting moiety peptide can be produced as part of a larger protein, for example as the N-terminus residues, and cleaved from that larger protein. Polypeptides suitable as “backbone” moieties are also suitable as target moieties, if they have sufficient affinity for the target organism. Considerations described are thus appropriate to consideration of a targeting moieties.

Targeting moieties need not be limited to peptide compositions, but can be lectins, polysaccharides, steroids, and metalloorganic compositions. Targeting moieties can be comprised of compositions that are composed both of amino acids and sugars, such as mucopolysaccharides. A useful targeting moiety can be partially lipid and partially peptide in nature, such as low density lipoprotein. Serum lipoproteins especially high density and low density lipoproteins (HDL and LDL) can bind to bacterial surface proteins (Emancipator, K. et al., Infect. Immun. 60:596-601, 1992). HDL and especially reconstituted HDL neutralizes bacterial lipopolysaccharide both in vitro and in vivo (Wurfel M M et al., J. Exp. Med. 181:1743-1754, 1995). Endogenous LDL can protect against the lethal effects of endotoxin and Gram negative infection (Netea, M., et al., J. Clin. Invest. 97:1366-1372, 1996). The appropriate binding features of the lipoproteins to bacterial surface components can be identified by methods of molecular biology known in the art, and the binding feature of lipoproteins can be used as the targeting moiety in photosensitizer compositions of the present invention.

Molecules, e.g., peptides, other than antibodies and members of a high affinity ligand pairs, can be used to target a photosensitizer composition according to the invention to a target organism. Targeting moieties can be modified or refined. Once an example of a targeting moiety of reasonable affinity has been provided, one skilled in the art can alter the disclosed structure (of a polylysine polypeptide, for example), by producing fragments or analogs, and testing the newly produced structures for modification of affinity or specificity. Examples of methods which allow the production and testing of fragments and analogs are discussed in U.S. Pat. No. 6,462,070.

E. Backbones

Photosensitizer compositions according to the invention include those in which a “backbone” moiety, such as a polyamino acid, is linked to a photosensitizer and/or to a binder and/or to a targeting moiety. Additionally, the backbone can itself be a targeting moiety, e.g. polylysine.

Inclusion of a backbone in a composition with a photosensitizer and/or binder and/or targeting moiety can provide a number of advantages, including the provision of greater stoichiometric ranges of photosensitizers and/or binders and/or targeting moieties coupled per backbone. If the backbone possesses intrinsic affinity for a target organism, the affinity of the composition can be enhanced by coupling to the backbone. Furthermore, the specific range of organisms that can be targeted with one composition can be expanded by coupling two or more different targeting moieties to a single photosensitizer-backbone composition.

Peptides useful in the methods and compounds of the invention for design and characterization of backbone moieties include poly-amino acids which can be homo- and hetero-polymers of L-, D-, racemic DL- or mixed L- and D-amino acid composition, and which can be of defined or random mixed composition and sequence. Examples of naturally-occurring peptides with mixed D and L amino acid residues include bacitracin and tyrocidin. These peptides may be modeled after particular natural peptides, and optimized by the technique of phage display and selection for enhanced binding to a chosen target, so that the selected peptide of highest affinity is characterized and then produced synthetically.

Further modifications of functional groups can be introduced for purposes, for example, of increased solubility, decreased aggregation, and altered extent of hydrophobicity. Examples of non-peptide backbones include nucleic acids and derivatives of nucleic acids such as DNA, RNA and peptide nucleic acids; polysaccharides and derivatives such as starch, pectin, chitins, celluloses and hemi-methylated celluloses; lipids such as triglyceride derivatives and cerebrosides; synthetic polymers such as polyethylene glycols (PEGs) and PEG star polymers; dextran derivatives, polyvinyl alcohols, N-(2-hydroxypropyl)-methacrylamide copolymers, poly(DL-glycolic acid-lactic acid); and compositions containing elements of any of these classes of compounds.

### III. Administration of the Photosensitizer Compositions of the Invention

The photosensitizer compositions of the invention can be delivered to a subject in a free form, i.e., in solution. Alternatively the compositions can be delivered in various formulations including, but not limited to, liposome, peptide-bound, polymer-bound, or detergent-containing formulations. Those of ordinary skill in the art are well able to generate and administer such formulations. The composition should be soluble under physiological conditions, in aqueous solvents containing appropriate carriers or excipients, or in other systems, such as liposomes, that may be used to administer the conjugate to a subject.

Photosensitizer compositions that are somewhat insoluble in an aqueous solvent can be applied in a liposome, or a time release fashion, such that illumination can be applied intermittently using a regimen of periods of illumination alternating with periods of non-illumination. Other regimens contemplated are continuous periods of lower level illumination, for which a time-release formulation is suitable. !A composition of the present invention can be administered by a variety of methods known in the art, including orally and topically. In one aspect, a photosensitizer composition of the invention may be administered parenterally. The phrase “administered parenterally” as used herein means modes of administration other than oral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

As will be appreciated by the skilled artisan, the route and/or mode of administration will vary depending upon the desired results. A photosensitizer composition according to the invention can be contained in a pharmaceutically acceptable excipient or carrier. Included, without limitation, are any and all solvents, dispersion media, coatings, antibacterial and antifungal agents, isotonic and absorption delaying agents, and the like that are physiologically compatible. The use of such media and agents for pharmaceutically active substances is well known in the art. Preferably, the carrier is suitable for oral, intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

In one aspect, the carrier may protect the compound against rapid release, for example, a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In another aspect of the invention, the photosensitizer compositions can be administered by combination therapy, i.e., combined with other agents. For example, the combination therapy can include a composition of the present invention with at least one other photosensitizer, at least one antibiotic, or other conventional therapy.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

One of ordinary skill in the art can determine and prescribe the effective amount of the pharmaceutical composition as needed. For example, one could start doses of the known or novel photosensitizer composition levels lower than that indicated in order to achieve the desired therapeutic effect and gradually increase the dosage until the desired effect is achieved. For example, the dosage may range from 0.1 mg/kg to 10 mg/kg depending on the therapeutic agent used.

The iterations delineated above are not intended as limiting with respect to the nature of the conjugate photosensitizer compositions of the invention, or to a particular route of the administration.

### IV. Photoactivation

Typically, administration of a photosensitizer composition according to the invention is followed by a sufficient period of time to allow accumulation thereof at the target site. Upon encountering the pathogen or a host cell infected with the pathogen, the enzyme cleavage site of the linker is cleaved by enzymes produced by the pathogen. Of note, the enzymes can be secreted by, can be internal to, or can reside on the surface of the pathogen. As a result, the photosensitizers are no longer quenched. The photosensitizers can, subsequently, be activated by irradiation. This is accomplished by applying light of a suitable wavelength and intensity, for an effective length of time, at the site of the inflammation. As used herein, “irradiation” refers to the use of light to induced a chemical reaction of a photosensitizer.

Photoactivating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art.

Irradiation of the appropriate wavelength for a given compound may be administered by a variety of methods. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination. Delivery of the light to a recessed, or otherwise inaccessible physiological location can be facilitated by flexible fiber optics (implicit in this statement is the idea that one can irradiate either a broad field, such as the lung or a lobe of the lung, or a narrow field where bacterial cells may have localized). The source of the light needed to inactivate the bacteria can be an inexpensive diode laser or a non-coherent light source.

The photosensitizer compositions of the invention should be stable during the course of at least a single round of treatment by continued or pulsed irradiation, during which the photosensitizer within the composition would, preferably, be repeatedly excited to the energized state, undergoing multiple rounds of generation of singlet oxygen.

The suitable wavelength, or range of wavelengths, will depend on the particular photosensitizer(s) used, and can range from about 350 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, photosensitizers with strong absorbances in the range of about 600 nm to about 900 nm are optimal for PDT. For photoactivation, the wavelength of light is matched to the electronic absorption spectrum of the photosensitizer so that the photosensitizer absorbs photons and the desired photochemistry can occur. Wavelength specificity for photoactivation generally depends on the molecular structure of the photosensitizer. Photoactivation can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photosensitizer can alter these values. In general, photosensitizers with longer absorbing wavelengths and higher molar absorption coefficients at these wavelengths are more effective photodynamic agents.

The light for photoactivation can be produced and delivered to the site of inflammation by any suitable means known in the art. Photoactivating light can be delivered to the site of inflammation from a light source, such as a laser or optical fiber. Preferably, optical fiber devices that directly illuminate the site of inflammation deliver the photoactivating light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of light-emitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of photosensitizer composition, and light activating the photosensitizer composition, is administered in an amount sufficient to produce a phototoxic species. For example, where the photosensitizer is chlorinₑ₆, administration to humans is in a dosage range of about 0.1 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 2-500 J/cm², preferably about 5 to about 50 J/cm², and more preferably about 5 to about 10 J/cm². The fluence rate is in the range of about 20 to about 500 mw/cm², preferably about 50 to about 300 mw/cm² and more preferably about 100 to about 200 mw/cm². There is a reciprocal relationship between photosensitizer compositions and light dose, thus, determination of suitable wavelength, light intensity, and duration of illumination is within ordinary skill in the art.

Dosage regimens are adjusted to provide the optimum desired response (e.g., a therapeutic response). For example, a single bolus may be administered, several divided doses may be administered over time, or the dose may be proportionally reduced or increased as indicated by the exigencies of the therapeutic situation.

Irradiation of the appropriate wavelength for a given compound may be administered by a variety of wavelengths. Methods for irradiation include, but are not limited to, the administration of laser, nonlaser, or broad band light. Irradiation can be produced by extracorporeal or intraarticular generation of light of the appropriate wavelength. Light used in the invention may be administered using any device capable of delivering the requisite power of light including, but not limited to, fiber optic instruments, arthroscopic instruments, or instruments that provide transillumination.

The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorine, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

### V. Targets

A. Subjects

The subject is a living animal or human (e.g., host) carrying an unwanted organism (e.g., pathogen), that is, an organism that is a target for photodynamic therapy. The subject can be a mammal, such as a human or a non-human mammal (e.g., a dog, cat, pig, cow, sheep, goat, horse, rat, or mouse). The subject may be further immune deficient; presently or previously undergoing treatment for cancer (e.g., by chemotherapy or radiation therapy); or presently or previously undergoing antibiotic therapy or an immunosuppressive therapy.

B. Pathogens

An organism that is targeted for destruction by the methods and compositions described herein is an unwanted organism, unwanted in that it infects a host organism (or a cell thereof) and causes or aggravates a disease or disorder in that host.

Target organisms can be cellular. Such target organisms include at least a boundary cell membrane and are capable of energy production, nucleic acid synthesis, and contain ribosomes and are capable of ribosomal protein synthesis. Cells can be unicellular or multicellular, and said unicellular organisms can be prokaryotic or eukaryotic.

Prokaryotic target organisms include bacteria, which bacteria can be Gram negative or Gram positive, or which are lacking cell walls. The Gram stain basis of distinguishing bacteria, based on whether or not cells of a specific strain or species of bacteria take up a stain, or are stained with the counterstain only, is known to those of skill in the art.

Gram negative, largely β lactamase-producing, bacterial genera suitable as target organisms include Neisseria, Pasteurella, Proteus, Pseudomonas, Streptophomonas, Burkholderia, Acinetobacter, Serratia, Salmonella, Enterobacter, Escherichia, Haemophilus, and Klebsiella. Streptophomonas maltophilia, Burkholderia cepacia, and Acinetobacter baumannii are, for example, common colonizers of patients in an intensive care setting. Gram positive bacterial genera suitable as target organisms include Staphylococcus and Enterococcus.

Other bacterial pathogens to be contemplated herein as “unwanted organisms” and, thus, to be targeted for destruction, include, without limitation, Mycobacterium tuberculosis, Leishhmania, Mycobacterium leprae, and Sheigella. Leishmania do not produce β lactamase. Rather, they produce surface metalloproteinase gp63, which can, for example, cleave the heptapeptide AYLKKWV. Thus, the latter polypeptide may serve as a suitable enzyme cleavage site within a linker in a photosensitizer composition according to the invention.

In specific embodiments, pathogens to be targeted by the compositions and methods of the present invention can be found on any light-accessible surfaces or in light-accessible areas, for example, in human and animal subjects. In the cases of humans and animals, infections of the epidermis, oral cavity, nasal cavity, sinuses, ears, lungs, urogenital tract, and gastrointestinal tract are light accessible. Epidermal infections include subcutaneous infections, especially localized lesions, which infections are light-accessible. Infections of the peritoneal cavity, such as those resulting from burst appendicitis, are light accessible via at least laparoscopic devices. A variety of skin infections which are refractory to antibiotics or long-term antifungal treatment, for example, dermatophycoses of the toenail, are suitable for photodynamic therapy using the methods and compositions of the invention.

Lung infection can occur with a variety of bacterial genera and species, which include the pseudomonads, which are the primary cause of death of cystic fibrosis patients, Klebsiella, and can also occur with a variety of virus strains. As pathogens of the lung are increasingly resistant to classical antibiotic therapies, photodynamic therapy with the compositions of the instant invention offer an alternative method for eliminating these unwanted organisms that is independent of the microbial mechanisms of resistance.

Additional epidermal infections and infections of deeper tissues arise from bums, scrapes, cuts, and puncture wounds. PDT with the compositions of the instant invention is useful for sterilization of such potential infectious sites, which can rapidly lead to toxic shock, a frequent concomitant of bullet wounds, and for treating the sites to eliminate or reduce unwanted infectious organisms. A major cause of infection in wounds, especially bums, is the Gram negative aerobic bacterium Pseudomonas. This organism produces an exotoxin which has been shown to retard wound healing. Multi-antibiotic resistant P. aeruginosa strains are becoming a significant problem, especially in bums units of large hospitals. Pseudomonads also produce fulminating infections of the cornea. Escherichia coli along with Staphylococcus aureus are the two most common bacteria in infected wounds.

Other sites of unwanted target organisms include the urogenital tract, the peritoneal cavity, the inner and outer ear, the nasal cavity and the gastrointestinal tract. Infectious sites of proliferation of unwanted target organisms in tissues of mesothelial and endothelial origin are also accessible to PDT by minimally invasive techniques.

In other specific embodiments, areas of infection are not light-accessible. Such areas can be accessed, for example, with the use of light-emitting probes or catheters. Thus, delivery of the light to a recessed, or otherwise inaccessible physiological location can be facilitated by flexible fiber optics (implicit in this statement is the idea that one can irradiate either a broad field, such as the lung or a lobe of the lung, or a narrow field where bacterial cells may have localized). The source of the light needed to inactivate the bacteria can be an inexpensive diode laser or a non-coherent light source.

The pathogens to be targeted by the compositions and methods of the present invention may be naturally or non-naturally occurring. Non-naturally occurring pathogens comprise pathogens recombinantly engineered, for example, to exhibit resistance to certain standard antibodies. In a situation of bioterrorism, for example, one might envision a pathogen that does not naturally produce β lactamase being engineered to produce the latter. Recombinantly engineering a naturally occurring pathogen to exhibit multiple antibody resistance would yield a highly virulent strain difficult to combat by standard treatment measures (such as penicillin).

These and other bacterial groups and genera not listed here will be recognized by the skilled artisan as suitable target bacteria for the compositions of the invention. Thus, the above lists are used to illustrate applications of the present invention to major groups of suitable target organisms, but not to delimit the invention to the species, genera, families, orders or classes so listed.

The pathogen may be contained within a host cell, such as a phagocyte (e.g., a macrophage). Further, within that cell, the pathogen may be contained (wholly or partly) within a vacuole, vesicle, or organelle.

The present invention is additionally described by way of the following illustrative, non-limiting Examples that provide a better understanding of the present invention and of its many advantages.

### Examples

### Example 1

### Preparation of Conjugates Comprising Polymer, β-lactam Moiety and Photosensitizer

In one approach, the synthesis of the conjugates is based on cephalosporin, the most often used β-lactam. It is conceivable to develop penem or carbapenem derivatives subsequently.

In the following, the photosensitizer (a porphyrin molecule with at least one propionic side chain) is represented by PS—CH₂—CH₂—COOH. The polymer used in the synthetic routes shown below is a linear or branched poly(ethylene glycol) with propionic acid groups (PEG-CH₂—CH₂—COOH) (Senter, P. D., et al. (1995) Bioconjug. Chem. 6:389-394). However, the chemistry is applicable to similar polymeric materials containing available carboxylic side chains. In order to be released upon enzymatic hydrolysis, the porphyrin molecule is preferably linked at the 3′-position of the cephalosporin. The cephalosporin-porphyrin moiety obtained can then be conjugated to the polymer using the amino group on the β-lactam ring.

The preparation of three different conjugates is proposed, where the porphyrin and cephalosporin are linked via an ester:

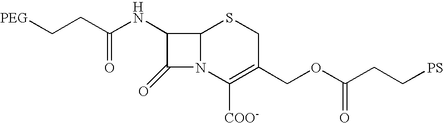

or via a carbamate group:

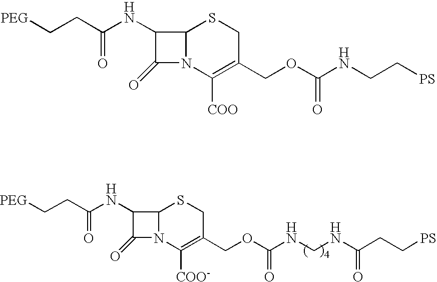

The preparation of a cephalosporin-prophyrin ester comprises the following steps:

A. Protection of the amino-group in the β-lactam ring

There are several ways to protect the amino group. One is represented below (Hanessian, S., et al. (1993) Can. J. Chem. 71:896-906):

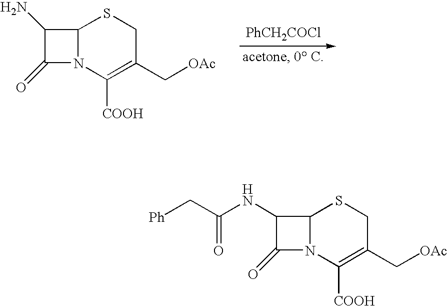

Protected cephalosporin derivatives are commercially available. Other protecting groups include (Albrecht, H. A., et al., (1990) J. Med. Chem. 33:77-86; Albrecht, H. A., et al. (1991) J. Med. Chem. 34:2857-2864; Alexander, R. P., et al. (1991) Tetrahedron Lett. 32:3269-3272):

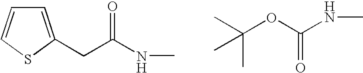

For example, the following molecule (which comes with a protected amino group) is called cephalothin.

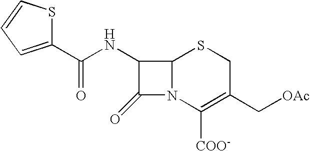

B. Binding of the porphyrin at the 3′-position of the cephalosporin via an ester function
i. Through a diazomethyl intermediate (Mobashery, S., et al. (1986) J. Biol. Chem. 261:7879-7887)

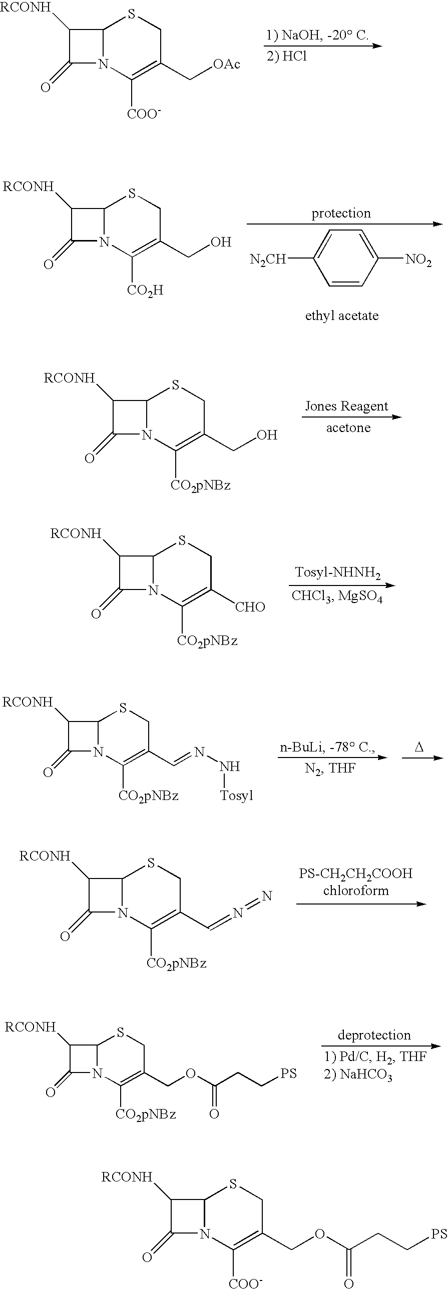

In this scheme, pNBz=para-nitro-benzyl.
ii. Through a halogenated intermediate (Mobashery, S., et al. (1986) J. Biol. Chem. 261:7879-7887)

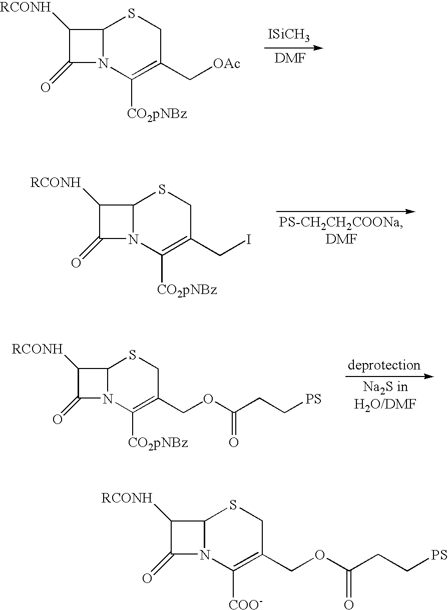

iii. Through a hydroxymethyl intermediate (Hanessian, S., et al. (1993) Can. J. Chem. 71:896-906)

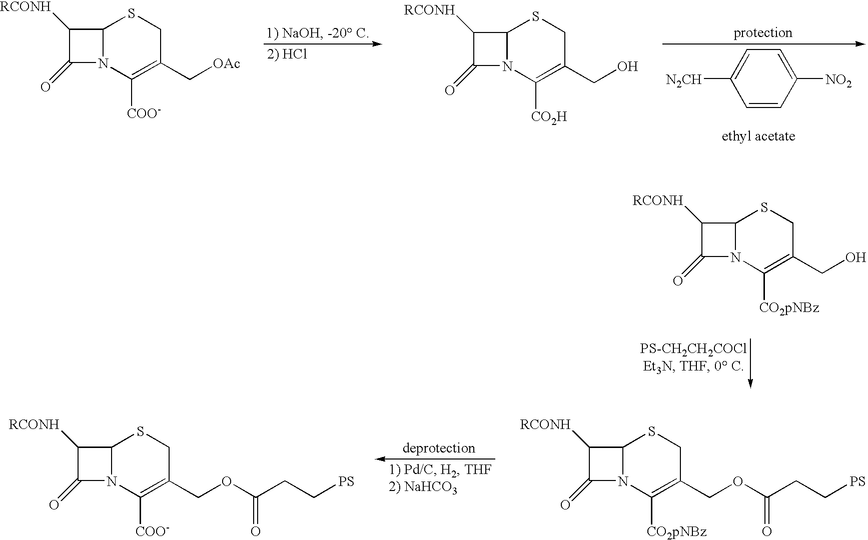

C. Deprotection of the amino-group in the B-lactam ring (Albrecht, H. A., et al. (1991) J. Med. Chem. 34:669-675)

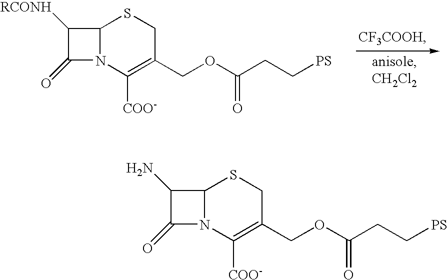

Deprotection of the amino group is also very often carried out using Penicillin-G amidase (PGA) (Vrudhula, V. M., et al. (1995) J. Med. Chem. 38:1380-1385).

D. Conjugation of the cephalosporin-porphyrin moiety to a polymer (Senter, P. D., et al. (1995) Bioconjug. Chem. 6:389-394)

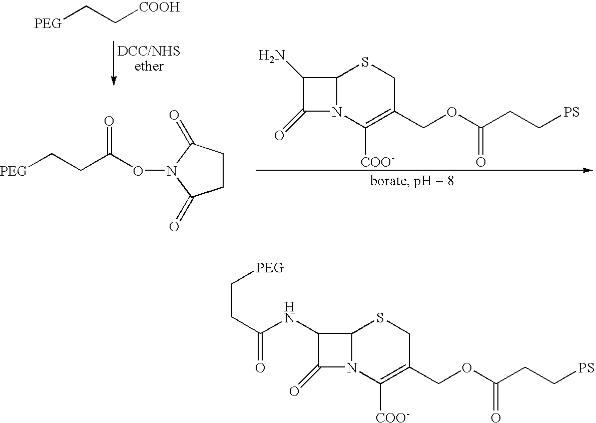

The preparation of a cephalosporin-porphyrin carbamate comprises the following steps:

A. Protection of the amino-group in the β-lactam ring (see above)

B. Binding of the porphyrin at the 3′-position of the cephalosporin via a carbamate
i. Direct coupling between the porphyrin and cephalosporin (Alexander, R. P., et al. (1991) Tetrahedron Lett. 32:3269-3272; Rodrigues, M. L., et al. (1995) Chem. & Biol. 2:223-227; Smith, K. M., et al. (1987) Heterocycles 26:1947-1963)

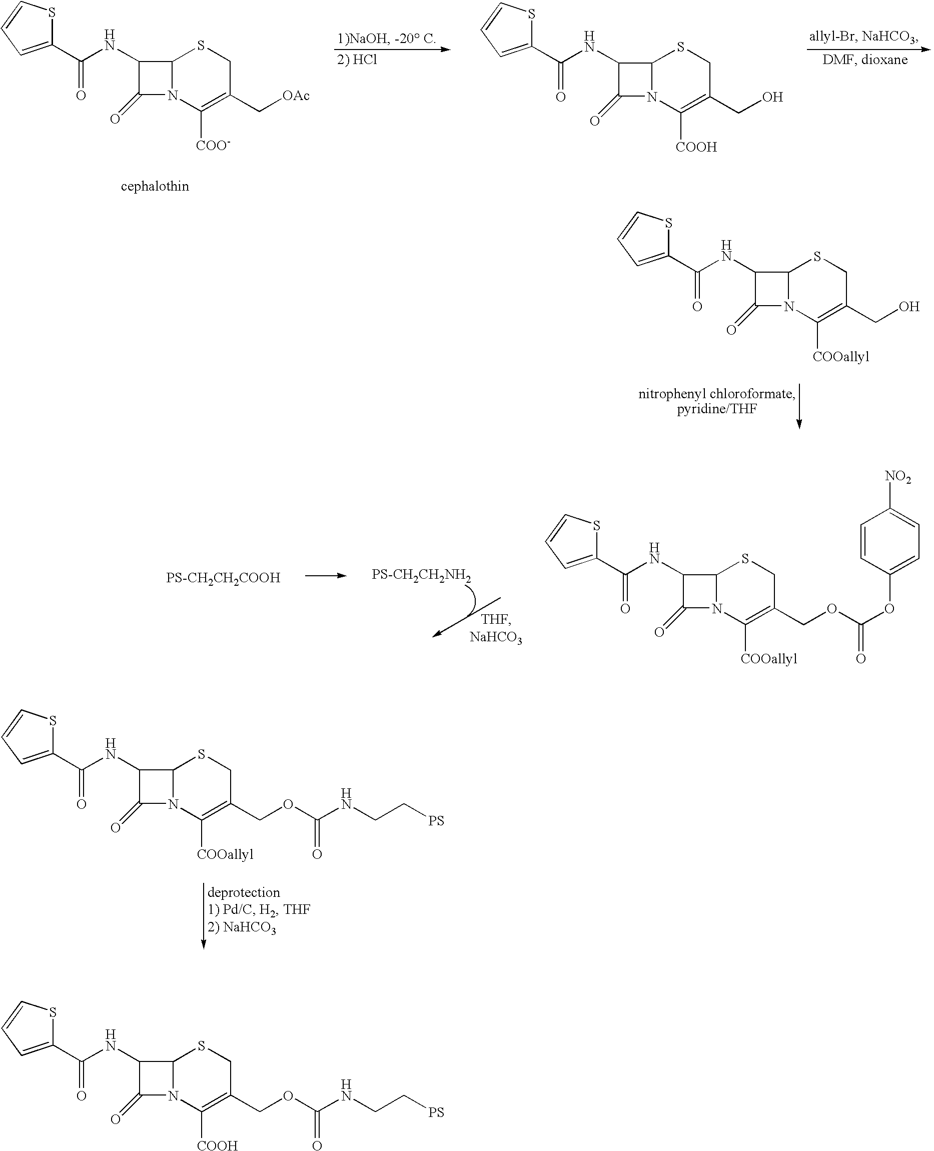

ii. Coupling through a linker (Alexander, R. P., et al. (1991) Tetrahedron Lett. 32:3269-3272; Rodrigues, M. L., et al. (1995) Chem. & Biol. 2:223-227; Boutorine, A. S., et al. (1996) J. Am. Chem. Soc. 118:9469-9476)

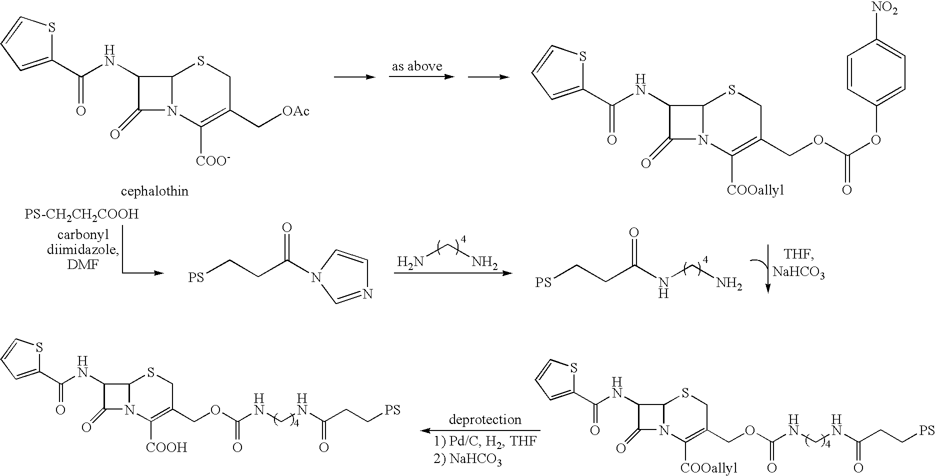

C. Deprotection of the amino-group in the β-lacatam ring (see above)

D. Conjugation of the cephalosporin-porphyrin moiety to a polymer (Senter, P. D., et al. (1995) Bioconjug. Chem. 6:389-394) (see above)

Of additional note, if, after these chemical modifications, the cephalosporin derivatives described above retain their properties as substrates for β-lactamases, one can expect to observe the enzyme-dependent release of three different porphyrin moieties:

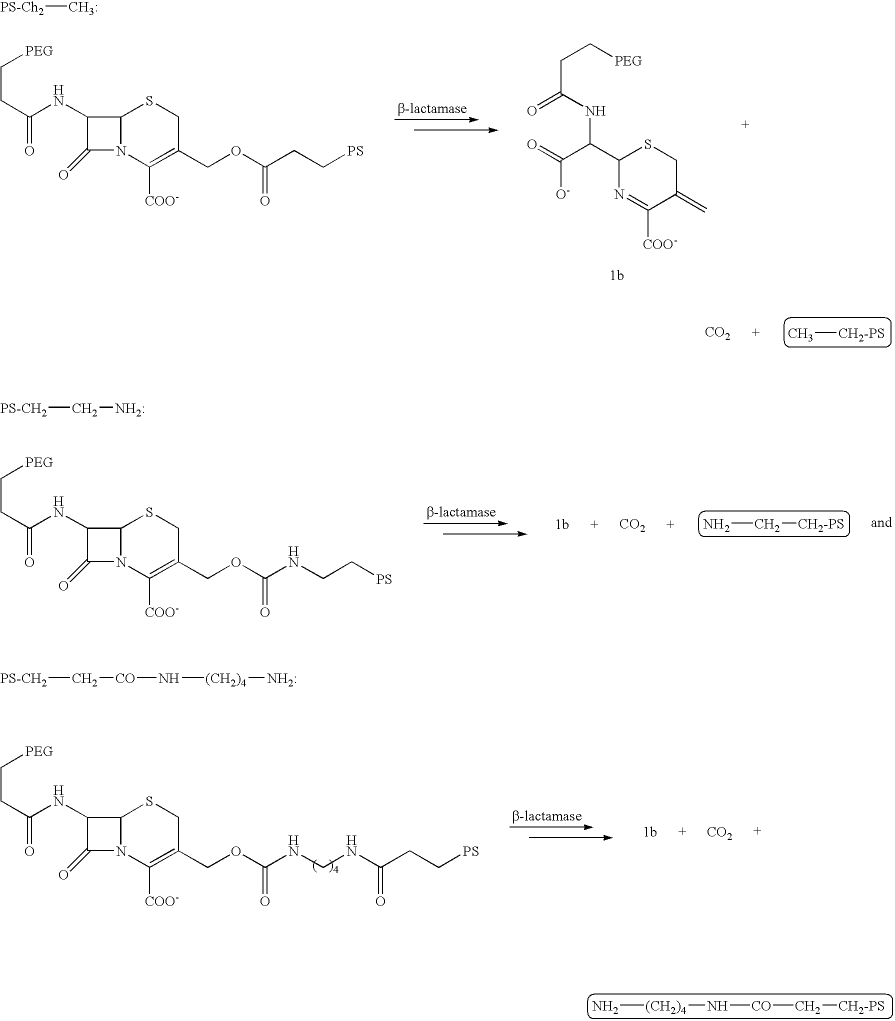

### Example 2

### Development of Carbamate-Linked Photosensitizer, Inactive (with or without Light) While Linked and Light-Activatable Only When Released by the β-lactamase Enzyme-Mediated Cleavage

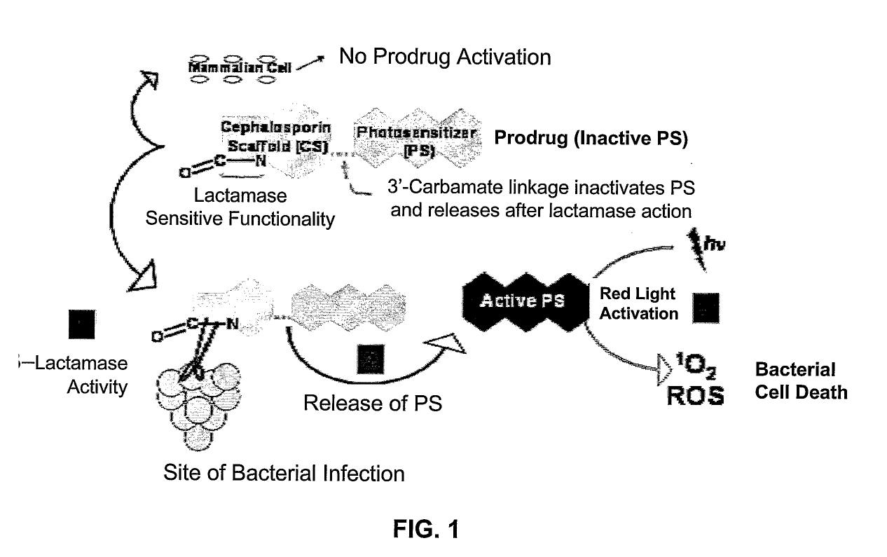

Unlike conventional antibiotics, where hydrolysis of the beta-lactam ring by β-lactamases causes inactivation, the lactam ring opening of the prodrugs releases the photosensitizer and make it light-activatable for photokilling (FIG. 1).

### Synthesis

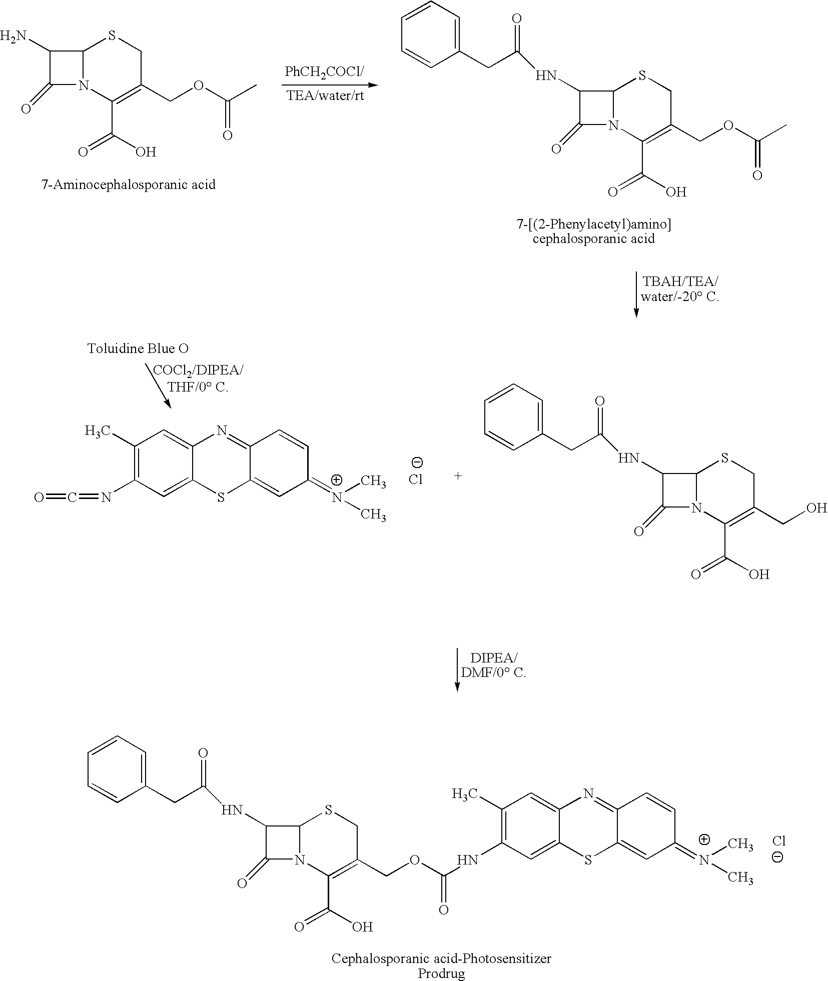

Commercially available 7-aminochephalosporanic acid was reacted with phenylacetyl chloride under Shotten-Baumann reaction conditions to achieve an amino protected chephalosporin molecule. This was further de-esterified using tetrabutylammonium hydroxide as a base to yield easily functionalizable hydroxy end group on cephalosporin. The last step of the synthesis was achieved in a one-pot reaction sequence. Toluidine Blue O (TBO) was converted into its isocynate derivative in the presence of diphosgene. The carbamate-linked prodrug was obtained by adding Cephalosporin derivative to the same reaction mixture.

### Synthesis of 7-[(2-phenylacetyl)amino]cephalosporanic acid

To a stirred mixture of sodium bicarbonate (2.1 g, 25 mmol) in water (40 ml) and acetone (30 ml), added 7-(phenylacetyl)amino cephalosporanic acid. Stirred this solution for nearly 15 min in ice bath and slowly added phenylacetyl chloride (2.5 ml, 20 mmol) over the period of 30 min. This reaction mixture was stirred overnight and acidified to pH 2.0 with 1N hydrochloric acid. Precipitates obtained were extracted with dichloromethane and washed with water. Dried over magnesium sulphate and solvent evaporated to give off-white solid. The solid sample was stirred overnight in diethyl ether and filters to obtain crude product in 80% yield.

### Synthesis of 7-[(2-phenylacetyl)amino]3-hydrodxymethy cephalosporanic acid

To a suspension of 7-[(2-phenylacetyl)amino]cephalosporanic acid (0.5 g, 1.28 mmol) in a a mixture of methane (4 ml) and water (2.5 ml), triethylamine (0.21 ml, 1.54 mmol) was added in 15 min at 0-5° C. To this solution, tetrabutylammonium hydroxide (30% solution in water, 1.53 g, 1.92 mmol) was added at −18° C. in 30 minutes. The reaction mixture was maintained at −18° C. for nearly 7.0 h and acidified to pH 5.0 using glacial acetic acid. Purification was done using C-18 reverse phase column and pure product was obtained as white solid in 67% yield.

### Synthesis of Cephalosporanic Acid-Toluidine BlueO Prodrug

To a magnetically stirred suspension of toludine blue O (0.1 g, 0.33 mmol) in anhydrous THF (3 ml) under nitrogen was added a solution of tricholoromethyl chloroformate (19.7 μl, 0.164 mmol) over activated charcoal as a catalyst. The reaction mixture was stirred at 55° C. for 30 min. Progress of reaction was monitored using mass spectroscopy for formation of isocynate derivative of toludine blue O. Cooled the flask to room temperature and added a solution of 7-[(2-phenylacetyl)amino]3-hydrodxymethy cephalosporanic acid (0.15 g, 0.33 mmol) in anhydrous dichloromethane (1 ml). The reaction flask was cooled to 0° C. and slowly added diisopropylethylamine (57.0 μl, 0.33 mmol). Stirred for 3.0 h and purified using C18 column with acetonirile and water as eluting solvents. Pure product obtained as a blue solid in 25% yield.

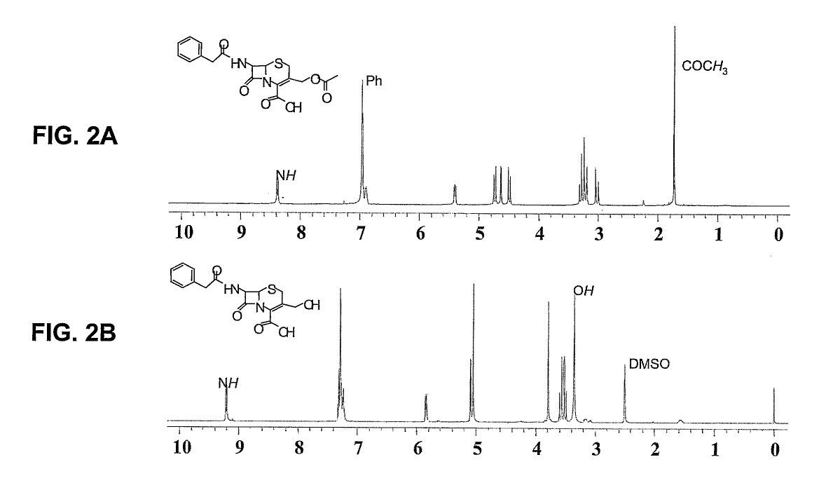

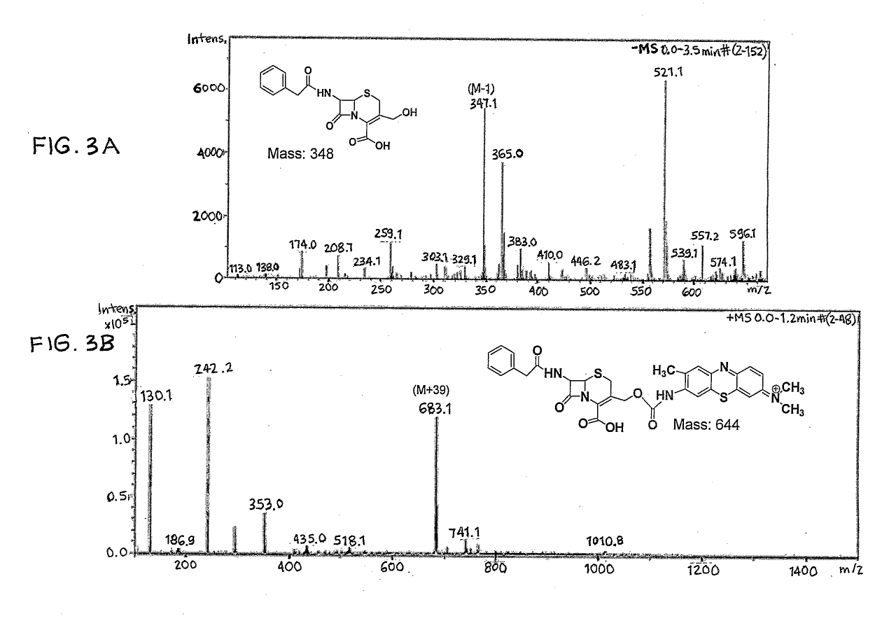

¹H NMR spectra were obtained for 7-[(2-phenylacetyl)amino]cephalosporanic acid in CDCl₃ as a solvent, as well as for 7-[(2-phenylacetyl)amino]3-hydrodxymethy cephalosporanic acid in DMSO-d₆ as a solvent (FIG. 2). MS spectra were obtained for 7-[(2-phenylacetyl)amino]3-hydrodxymethy cephalosporanic acid and cephalosporanic acid-toluidine blue O prodrug (FIG. 3).

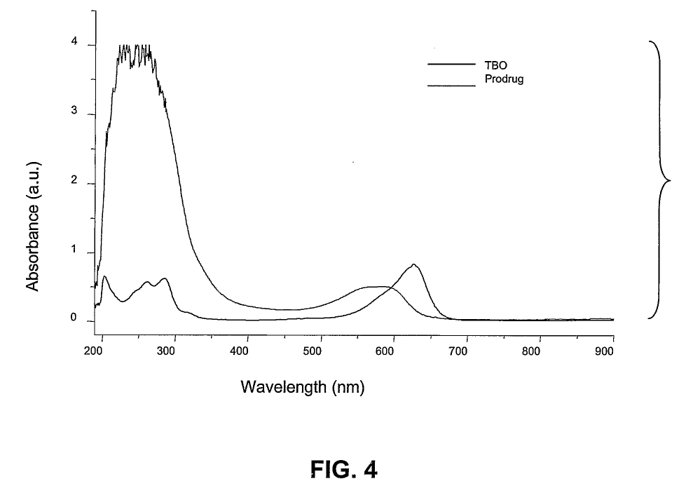

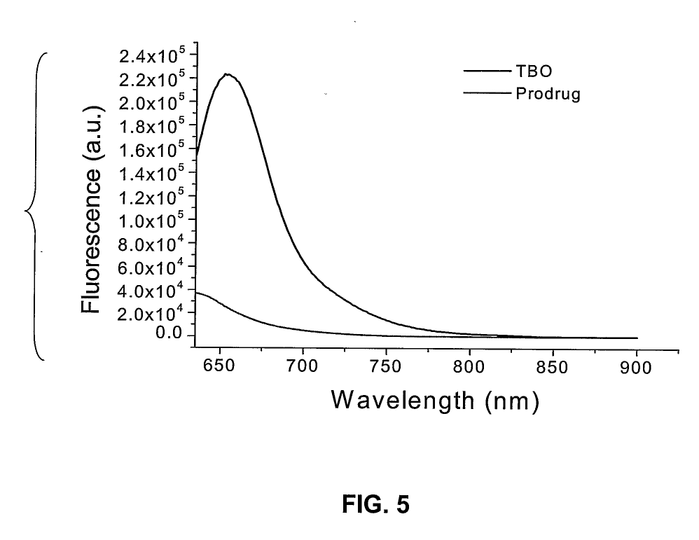

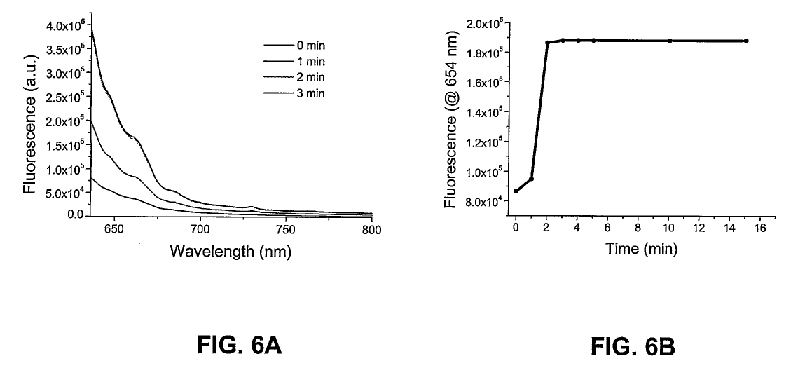

UV-visible spectra revealed blue shift in the absorption spectra of the prodrug, indicating extended conjugation, as well as quenching, of carbamate linked TBO photosensitizer (FIG. 4). Fluorescence spectra revealed nearly an 8-fold reduction in fluorescence emission maxima at 635 nm excitation, indicating quantitative quenching of the photosensitizer upon conjugation with the cephalosporin moiety (FIG. 5). Enzyme-mediated cleavage of the prodrug The prodrug obtained was further studied for release of photosensitizer in presence of β-lactamase from Enterobacter cloacae. For the fluorescence emission study of the prodrug, the solvent employed was water, and the excitation wavelength 635 nm in the presence of beta-lactamase enzyme (from Enterobacter cloacae). Time-dependent fluorescence emission was also measured for photosensitizer release from the prodrug in the presence of enzyme. The results indicate an easy release and nearly 5-fold increase in excited stated properties within minutes of incubation of prodrug with enzyme (FIG. 6). Thus, the prodrug was synthesized and characterized. Furthermore, the prodrug showed quantitative quenching of the photosensitizer in the conjugated form. Additionally, the product demonstrated lactamase-specific activity.

1. A photosensitizer composition comprising a plurality of photosensitizers that are linked by one or more linkers, wherein said one or more linkers comprise an enzyme cleavage site for an enzyme of a pathogen and wherein said linked photosensitizers are present in an amount sufficient to quench photoactivation of said photosensitizers.

2. The photosensitizer composition of claim 1, wherein the enzyme cleavage site comprises a cephalosporin, a penicillin, a penem, a carbapenem, a monocyclic mobactem, a polypeptide cleavable by an enzyme of Leishmania, or a fragment thereof.

3. The photosensitizer composition of claim 2, wherein the enzyme cleavage site comprises a cephalosporin, a penicillin, or a fragment thereof.

4. The photosensitizer composition of claim 3, wherein the cephalosporin or penicillin fragment comprises a beta-lactam ring.

5. The photosensitizer composition of claim 3, wherein the enzyme cleavage site is a cephalosporin.

6. The photosensitizer composition of claim 5, wherein at least one photosensitizer is bound at the 3′ position of a cephalosporin.

7. The photosensitizer composition of claim 4, wherein the enzyme cleavage site is cleaved by a lactamase.

8. The photosensitizer composition of claim 1, wherein the pathogen is a Gram (+) bacterium.

9. The photosensitizer composition of claim 1, wherein the pathogen is a Gram (−) bacterium.

10. The photosensitizer composition of claim 1, wherein the pathogen is selected from the group consisting of Staphylococcus, Enterococcus, Enterobacter, Escherichia, Haemophilus, Neisseria, Klebsiella, Pasteurella, Proteus, Pseudomonas, Streptophomonas, Burkholderia, Acinetobacter, Serratia, and Salmonella spp.

11. The photosensitizer composition of claim 1, wherein the pathogen is selected from the group consisting of Staphylococcus aureus, Staphylococcus epidermis, Enterococcus faecalis, Enterococcus faecium, Escherichia coli, Haemophilus influenzae, Neisseria gonorrhea, Klebsiella pneumoniae, Pasteurella multocida, Proteus mirabilis, Pseudomonas aeruginosa, Stenotrophomonas maltophilia, Burkholderia cepacia, Acinetobacter baumannii, Enterobacter aerogines, Enterobacter cloacae, Serratia marcescens, Salmonella enterica, and Salmonella typhimurium.

12. The photosensitizer composition of claim 1, wherein the enzyme cleavage site comprises D-ala-D-ala (SEQ ID No. 1).

13. The photosensitizer composition of claim 12, wherein the enzyme cleavage site is cleaved by a dipeptidase.

14. The photosensitizer composition of claim 2, wherein the enzyme cleavage site comprises a polypeptide cleavable by an enzyme of Leishmania.

15. The photosensitizer composition of claim 14, wherein the enzyme of Leishmania is surface metalloproteinase gp63.

16. The photosensitizer composition of claim 15, wherein the polypeptide is the heptapeptide AYLKKWV (SEQ ID No. 2).

17. The photosensitizer composition of claim 1, wherein the photosensitizer is a porphyrin.

18. The photosensitizer composition of claim 17, wherein the porphyrin is selected from the group consisting of a porfimer sodium, hematoporphyrin IX, hematoporphyrin ester, dihematoporphyrin ester, synthetic diporphyrin, O-substituted tetraphenyl porphyrin, 3,1-meso tetrakis porphyrin, hydroporphyrin, benzoporphyrin derivative, benzoporphyrin monoacid derivative, monoacid ring derivative, tetracyanoethylene adduct of benzoporphyrin, dimethyl acetylenedicarboxylate adduct of benzoporphyrin, δ-aminolevulinic acid, benzonaphthoporphyrazine, naturally occurring porphyrin, ALA-induced protoporphyrin IX, synthetic dichlorin, bacteriochlorin tetra(hydroxyphenyl)porphyrin, purpurin, octaethylpurpurin derivative, etiopurpurin, tin-etio-purpurin, porphycene, chlorin, chlorin e₆, mono-l-aspartyl derivative of chlorin e₆, di-l-aspartyl derivative of chlorin e₆, tin(IV) chlorin e₆, meta-tetrahydroxyphenylchlorin, chlorin e₆ monoethylendiamine monamide, verdin, zinc methyl pyroverdin, copro II verdin trimethyl ester, deuteroverdin methyl ester, pheophorbide derivative, pyropheophorbide, texaphyrin, lutetium (III) texaphyrin, and gadolinium(III) texaphyrin.

19. The photosensitizer composition of claim 1, wherein the photosensitizer is a photoactive dye.

20. The photosensitizer composition of claim 19, wherein the photoactive dye is selected from the group consisting of a merocyanine, phthalocyanine, chloroaluminum phthalocyanine, sulfonated aluminum PC, ring-substituted cationic PC, sulfonated AlPc, disulfonated or tetrasulfonated derivative, sulfonated aluminum naphthalocyanine, naphthalocyanine, tetracyanoethylene adduct, crystal violet, azure β chloride, benzophenothiazinium, benzophenothiazinium chloride (EtNBS), phenothiazine derivative, rose Bengal, toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, and thionine.

21. The photosensitizer composition of claim 1, wherein the photosensitizer is selected from the group consisting of a Diels-Alder adduct, dimethyl acetylene dicarboxylate adduct, anthracenedione, anthrapyrazole, aminoanthraquinone, phenoxazine dye, chalcogenapyrylium dye, cationic selena, tellurapyrylium derivative, cationic imminium salt and tetracycline.

22. The composition of claim 1, wherein the composition comprises a plurality of the same photosensitizer.

23. The composition of claim 1, wherein the composition comprises a targeting moiety.

24. The composition of claim 23, wherein the targeting moiety targets the composition to a pathogen or a host cell infected with a pathogen.

25. The composition of claim 24, wherein the infected host cell is a macrophage.

26. The composition of claim 23, wherein the targeting moiety comprises a liposome.

27. The composition of claim 23, wherein the targeting moiety comprises a peptide.

28. The composition of claim 27, wherein the peptide is a small anti-microbial peptide or an active fragment or analog thereof.

29. A pharmaceutical composition comprising a therapeutically effective amount of the composition of claim 1, and a pharmaceutically acceptable excipient or carrier.

30. A photosensitizer composition comprising a plurality of photosensitizers and one or more binders effective to quench photoactivation, wherein the photosensitizers are connected to the binder through a linker comprising an enzyme cleavage site for an enzyme of a pathogen.

31-53. (canceled)

54. A pharmaceutical composition comprising a therapeutically effective amount of the composition of claim 30, and a pharmaceutically acceptable excipient or carrier.

55. A photosensitizer composition comprising a backbone coupled to a plurality of photosensitizers and one or more binders effective to quench photoactivation, wherein the binders are connected to the backbone through a linker comprising an enzyme cleavage site for an enzyme of a pathogen.

56. A photosensitizer composition comprising a backbone coupled to a plurality of photosensitizers and one or more binders effective to quench photoactivation, wherein the photosensitizers are connected to the backbone through a linker comprising an enzyme cleavage site for an enzyme of a pathogen.

57-59. (canceled)

60. A method for decreasing the activity of a pathogen in a subject, said method comprising the steps of:
contacting the pathogen with a quenched photosensitizer composition comprising a plurality of photosensitizers that are linked by one or more linkers, wherein said one or more linkers comprise an enzyme cleavage site for an enzyme of a pathogen and wherein said linked photosensitizers are present in an amount sufficient to quench photoactivation of said photosensitizers;
cleaving one or more linkers to dequench the photosensitizer composition;
and light-activating the composition to produce a phototoxic species, thereby decreasing the activity of the pathogen in the subject.

61-86. (canceled)

87. A method for treating an infection in a subject, the method comprising administering to the subject a therapeutically effective amount of a photosensitizer composition of claim 1, and light-activating the composition to produce a phototoxic species, thereby treating the infection in the subject.

88-89. (canceled)

90. A packaged pharmaceutical comprising the photosensitizer composition of claim 1 and associated instructions for using said composition to decrease the activity of a pathogen in a subject.

91. A kit for decreasing the activity of a pathogen in a subject in need thereof comprising the photosensitizer composition of claim 1 and instructions for using the photosensitizer composition to decrease the activity of the pathogen in the subject.

92. A method of preparing a linked photosensitizer comprising the steps of: reacting an isocynate derivative of a photosensitizer with a cephalosporin, a penicillin, a penem, a carbapenem, a monocyclic mobactem, a polypeptide cleavable by an enzyme of Leishmania, or a derivative or fragment thereof.

93-99. (canceled)
